(19) United States
(12) Patent Application Publication
(10) Pub. No.: US 2008/0260817 A1
(43) Pub. Date: Oct. 23, 2008

(54) Compositions for the Treatment of Inflammation and Pain Using a Combination of a Cox-2 Selective Inhibitor and a Ltb4 Receptor Antagonist

(76) Inventors: Phenil Jayantilal Patel, Ann Arbor, MO (US); Po-Chang Chaing, Chesterfield, MO (US)

Correspondence Address: PHARMACIA CORPORATION;GLOBAL PATENT DEPARTMENT, POST OFFICE BOX 1027, ST. LOUIS, MO, 63006, US

(21) Appl. No.: 10/597,646

(22) PCT Filed: Jul. 11, 2005
(86) PCT No.: PCT/IB2005/002203

§ 371 Date: Jul. 8, 2008

Related U.S. Application Data
(60) Provisional application No. 60/590,076, filed on Jul. 22, 2004.

(51) Int. Cl. A61K 31/415; A61K 31/352; A61K 9/52; A61K 9/22; A61P 29/00

(52) U.S. Cl. 424/457; 514/456; 514/406; 424/472

(57) ABSTRACT

The present invention provides a therapeutic composition comprising a COX-2 selective inhibitor or a prodrug thereof and an LTB₄ receptor antagonist. A method is provided for the use of such composition in the treatment, prevention, or inhibition of inflammation, an inflammation-related disorder, pain, or a pain-related disorder.

### BACKGROUND OF THE INVENTION

1. Field of the Invention

The present invention relates to compositions and methods for the treatment and prevention of pain, pain-related disorders, inflammation, and inflammation-related disorders using a combination of a non-steroidal anti-inflammatory compound and a leukotriene B4 receptor antagonist.

2. Description of Related Art

Inflammatory mediators have been implicated to play a key pathogenic role in the initiation, propagation and continuation of pain and inflammation. Prostaglandins have been demonstrated to be important mediators of inflammation, as well as regulators of other significant functions not directly related to inflammation. Regulation of the production and activity of prostaglandins has been a common target of antiinflammatory drug discovery activities. However, common non-steroidal antiinflammatory drugs (NSAIDs) that are active in reducing the prostaglandin-induced pain and swelling associated with the inflammation process also have an effect, sometimes adverse, upon other prostaglandin-regulated processes not associated with the inflammation process.

The mechanism ascribed to many of the common NSAIDs is the modulation of prostaglandin synthesis by inhibition of cyclooxygenases that catalyze the transformation of arachidonic acid—the first step in the prostaglandin synthesis pathway. In the 1980's Needleman et al. discovered that two cyclooxygenases are involved in this transformation. These enzymes have been termed cyclooxygenase-1 (herein, “COX-1”) and cyclooxygenase-2 (herein, “COX-2”). See, Needleman, P. et al., J. Rheumatol., 24, Suppl. 49:6-8 (1997).

COX-1 has been shown to be a constitutively produced enzyme that is involved in many of the non-inflammatory regulatory functions associated with prostaglandins. COX-2, on the other hand, is an inducible enzyme having significant involvement in the inflammatory process. Inflammatory stimuli cause the production of COX-2, stimulating the release of prostanoids. The prostainoids, in turn, sensitize peripheral nociceptor terminals, thereby causing localized pain hypersensitivity. Many common NSAIDs are now known to be inhibitors of both COX-1 and COX-2 in vitro. Accordingly, when administered in sufficiently high levels, these NSAIDs affect not only the inflammatory consequences of COX-2 activity, but also the beneficial activities of COX-1.

Compounds that selectively inhibit COX-2 in an in vitro enzyme assay have been discovered. Advantages provided by the new COX-2 selective inhibitors include the capacity to prevent or reduce inflammation while avoiding harmful side effects associated with the inhibition of COX-1. Thus, COX-2 selective inhibitors have shown great promise for use in therapies, including those that require long term administration, such as for pain and inflammation control for arthritis.

Leukotrienes are compounds produced in mammals by the metabolism of arachidonic acid. Arachidonic acid is metabolized in mammals by two different pathways, one leading to the production of prostaglandins and thromboxanes, and the other to oxidative products known as leukotrienes. There are several different classes of leukotrienes, including leukotriene A4, leukotriene B4 (herein, “LTB₄”), leukotriene C₄, and leukotriene D₄. It is believed that LTB₄ is a mediator of inflammation and plays critical roles in diseases such as arthritis, psoriasis, myocardial infarction, irritable bowel disease, and many others.

In contrast to other leukotrienes, which primarily cause broncho-constriction and some proinflammatory effects, LTB₄ acts mainly as a chemoattractant and activator of leukocytes (Jennewein, H. M., et. al, Prog Respir. Res. Basel. Karger, 2001, vol 31, pp 121-125).

LTB₄ receptors are located on a variety of cells, primarily neutrophils but also macrophages, lymphocytes, eosinophils and lung epithelial cells. In polymorphonuclear leukocytes (PMNLs), LTB₄ causes chemoattraction, chemokinesis, oxidative burst and the upregulation or shedding of adhesion molecules as a prerequisite of adhesion. It also inhibits neutrophil apoptosis, thereby prolonging the inflammatory response.

LTB₄ has a key role in the pathophysiology of rheumatoid arthritis (herein, “RA”) (Alten R., et. al Ann Rheum Dis. 2004, February; 63(2):170-6). RA is an autoimmune disease characterized by joint inflammation, joint destruction, progressive disability, and premature death. Treatment of RA patients by administering to them disease-modifying antirheumatic drugs alone or in combination with other drugs may be useful to minimize the dire consequences of RA. Treatment with disease-modifying antirheumatic drugs may limit or prevent further disease progression (Hochberg, M. C., Scand J Rheumatol Suppl. 1999; 112:3-7).

U.S. Pat. No. 5,384,318 describes some substituted sulfonamides which are LTB₄ antagonists.

U.S. Pat. No. 5,246,965 describes some aryl ethers which are LTB₄ receptor antagonists.

U.S. Pat. No. 6,342,510 describes the combination of a COX-2 inhibitor and a LTB₄ receptor antagonist for the treatment of inflammation and inflammation-related disorders.

PCT Patent Application number WO 2004/047824 describes certain pharmaceutical compositions comprising an LTB₄ receptor antagonist having a hydroxy and a benzamidine group; and certain COX-2 inhibitors; and a pharmaceutically acceptable excipient.

There is a need for a therapy that inhibits pain and inflammation and also exhibits disease-modifying effects.

### SUMMARY OF THE INVENTION

Among the several embodiments of the present invention may be noted the provision of a therapeutic composition comprising at least one COX-2 selective inhibitor or a prodrug thereof and at least one LTB₄ receptor antagonist wherein the LTB₄ receptor antagonist comprises one or more compounds selected from the group consisting of 2-[(3S,4R)-3,4-dihydro-4-hydroxy-3-(phenylmethyl)-2H-1-benzopyran-7-yl]-4-(trifluoromethyl)benzoic acid; a pharmaceutically acceptable salt thereof; and mixtures thereof.

In another embodiment, the present invention provides a method for the treatment, prevention, or inhibition of inflammation, an inflammation-related disorder, pain, or pain-related disorder in a subject in need of such prevention, treatment, or inhibition, the method comprising administering to the subject a therapeutic composition comprising at least one COX-2 selective inhibitor or a prodrug thereof and at least one LTB₄ receptor antagonist wherein the LTB₄ receptor antagonist comprises one or more compounds selected from the group consisting of 2-[(3S,4R)-3,4-dihydro-4-hydroxy-3-(phenylmethyl)-2H-1-benzopyran-7-yl]-4-(trifluoromethyl)benzoic acid; a pharmaceutically acceptable salt thereof; and mixtures thereof.

Further scope of the applicability of the present invention will become apparent from the detailed description provided below. However, it should be understood that the following detailed description and examples, while indicating preferred embodiments of the invention, are given by way of illustration only since various changes and modifications within the spirit and scope of the invention will become apparent to those skilled in the art from this detailed description.

### BRIEF DESCRIPTION OF THE DRAWINGS

FIG. 1. Murine Air Pouch 2 Hour Zymosan

FIG. 2. Disease Severity

FIG. 3. Collagen-induced Arthritis Incidence

FIG. 4. Combination reduces Collagen-Induced Arthritis Incidence

### DETAILED DESCRIPTION

The following detailed description is provided to aid those skilled in the art in practicing the present invention. Even so, this detailed description should not be construed to unduly limit the present invention as modifications and variations in the embodiments discussed herein can be made by those of ordinary skill in the art without departing from the spirit or scope of the present inventive discovery.

The contents of each of the references cited herein, including the contents of the references cited within these primary references, are herein incorporated by reference in their entirety.

### a. DEFINITIONS

The following definitions are provided in order to aid the reader in understanding the detailed description of the present invention:

As used herein, the term “COX-1” refers to one of the two isoforms of the enzyme fatty acid cyclooxygenase called cyclooxygenase-1.

As used herein, the term “COX-2” refers to one of the two isoforms of the enzyme fatty acid cyclooxygenase called cyclooxygenase-2.

As used herein, the term “mg” refers to milligram.

As used herein, the term “g” refers to gram.

As used herein, the term “mpk” refers to milligrams per kilogram.

As used herein, the term “SLS” means sodium lauryl sulfate.

As used herein, the term “PVP” means polyvinylpyrrolidone.

As used herein, the terms “inflammation-related disorder” or “inflammation disorder” are meant to include, without limitation, each of the symptoms or disorders that are mentioned below. However, these terms are also meant to include any therapeutic condition in which inflammation or inflammation-related processes play a role.

As used herein, the terms “neoplasia” and “neoplasia disorder”, used interchangeably herein, refer to any new and abnormal cell growth, including one in which cell multiplication is uncontrolled and progressive. Neoplasms may be benign or malignant. Neoplasia also includes the term “cancer” and for purposes of the present invention; cancer is one subtype of neoplasia. As used herein, the term “neoplasia disorder” also encompasses other cellular abnormalities, such as hyperplasia, metaplasia and dysplasia. The terms neoplasia, metaplasia, dysplasia and hyperplasia can be used interchangeably herein and refer generally to cells experiencing abnormal cell growth.

Both of the terms, “neoplasia” and “neoplasia disorder”, refer to a “neoplasm” or tumor, which may be benign, premalignant, metastatic, or malignant.

As used herein, the terms “to prevent”, “preventing”, or “prevention” refer to any reduction, no matter how slight, of a subject's predisposition or risk for developing a disease or disorder including any of the following: (1) substantially preventing the onset of a clinically evident disorder or disease in a subject; (2) preventing the onset of a preclinically evident stage of a disease or disorder in a subject; or (3) substantially preventing the disorder or disease in a subject. This definition includes prophylactic treatment.

The term “inhibition” as used herein means a decrease in the severity of a disorder or disease as compared to that which would occur in the absence of the application of the present invention. This decrease in severity may result from a reduction in any one or combination of symptoms characteristic of the disease or disorder. For example, in the case of inflammation, this includes symptoms such as swelling, soreness, redness, stiffness, and others. On the cellular level this includes characteristics such as chemotaxis; the release of inflammatory mediators such as prostaglandins, chemokines, leukotrienes; cell infiltration; activation of immunological cells and others.

The phrase “therapeutically-effective” is intended to qualify the amount of each agent which will achieve the goal of improvement in disorder severity and the frequency of incidence relative to no treatment or treatment of each agent by itself, while avoiding adverse side effects typically associated with alternative therapies.

The term “subject” for purposes of treatment or prevention includes any human or animal who is susceptible to the disorder or disease. The subject can be a domestic livestock species, a laboratory animal species, a zoo animal or a companion animal. In one embodiment, the subject is a mammal. In an alternative embodiment, the mammal is a human being. In another embodiment, the subject is a companion animal such as a dog, a cat, or a horse.

The term “COX-2 selective inhibitor” embraces compounds which selectively inhibit the COX-2 enzyme over the COX-1 enzyme, and also include pharmaceutically acceptable salts and prodrugs of those compounds.

The selectivity of a COX-2 inhibitor varies depending upon the condition under which the test is performed and on the inhibitors being tested. For the purposes of this specification, the selectivity of a COX-2 inhibitor can be measured as a ratio of the in vitro or in vivo IC₅₀ value for inhibition of COX-1, divided by the IC₅₀ value for inhibition of COX-2 (COX-1 IC₅₀/COX-2 IC₅₀). A COX-2 selective inhibitor is any inhibitor for which the ratio of COX-1 IC₅₀ to COX-2 IC₅₀ is greater than 1. In one embodiment of the present invention, this ratio is greater than 2. In another embodiment of the present invention, the ratio is greater than 5. In yet another embodiment of the present invention, the ratio is greater than 10. In another embodiment, the ratio is greater than 50. In yet another embodiment of the present invention, the ratio is greater than 100. Please see Example number 1 for details regarding determination of COX-2 inhibitor activity and in vitro COX-2 selectivity.

For a COX-2 selective inhibitor, the term “IC₅₀” refers to the concentration of a compound that is required to produce 50% inhibition of enzyme activity in an in vitro enzyme assay as described hereinbelow. In one embodiment of the present invention, COX-2 selective inhibitors have an IC₅₀ of less than about 1 micromolar, alternatively less than about 0.5 micromolar, and alternatively less than about 0.2 micromolar.

For an LTB₄ receptor antagonist, the term “IC₅₀” refers to the concentration of a compound sufficient to inhibit 50% of specific ³H-LTB₄ binding in an in vitro assay as described hereinbelow. In one embodiment of the present invention, LTB₄ receptor antagonists have a IC₅₀ of less than about 1 micromolar, alternatively less than about 0.5 micromolar, and alternatively less than about 0.2 micromolar.

In one embodiment of the present invention, COX-2 selective inhibitors have an IC₅₀ of greater than about 1 micromolar. In another embodiment, the COX-2 selective inhibitors have an IC₅₀ greater than 20 micromolar. Inhibitors of the cyclooxygenase pathway in the metabolism of arachidonic acid used in the present method may inhibit enzyme activity through a variety of mechanisms. By the way of example, and without limitation, the inhibitors used in the methods described herein may block the enzyme activity directly by acting as a substrate for the enzyme.

As used herein, the term “leukotriene B₄ receptor antagonist” or “LTB₄ receptor antagonist” or “LTB₄ ra”, embraces compounds that selectively antagonize an LTB₄ receptor with an IC₅₀ of less than about 10 micromolar. In another embodiment of the present invention, LTB₄ receptor antagonists have an IC₅₀ of less than about 1 micromolar. Example 2 illustrates methods used in the present application for the determination of LTB₄ Receptor Antagonist Activity.

As used herein, the terms “treating”, “treatment”, “treated”, or “to treat,” mean to alleviate symptoms, eliminate the causation either on a temporary or permanent basis, or to alter or slow the appearance of symptoms or symptom worsening. The term “treatment” includes alleviation or elimination of causation of the symptoms associated with, but not limited to, any of the disorders or diseases or disorder-related or disease-related symptoms described herein.

Also included within the scope of the present invention are compounds that act as prodrugs of COX-2 selective inhibitors, or prodrugs of LTB₄ receptor antagonists. As used herein in reference to COX-2 selective inhibitors, the term “prodrug” refers to a chemical compound that can be converted into an active COX-2 selective inhibitor by metabolic or simple chemical processes within the body of the subject. One example of a prodrug for a COX-2 selective inhibitor is parecoxib, which is a therapeutically effective prodrug of the COX-2 selective inhibitor valdecoxib. An example of a preferred COX-2 selective inhibitor prodrug is sodium parecoxib. A class of prodrugs of COX-2 inhibitors is described in U.S. Pat. No. 5,932,598.

The term “pharmaceutically acceptable” is used herein to mean that the modified noun is appropriate for use in a pharmaceutical product.

As used herein, an “effective amount” means the dose or amount to be administered to a subject and the frequency of administration to the subject, which is readily determined by one having ordinary skill in the art, by the use of known techniques and by observing results obtained under analogous circumstances.

As used herein, the terms “in a subject in need of such treatment or prevention”, mean any subject who, at the time of presentation, exhibits the disease or disorder sought to be treated, or is at risk for developing a disease or disorder or symptoms of the disease or disorder.

### b. DETAILS

In accordance with the present invention, there is now disclosed a therapeutic composition comprising a COX-2 selective inhibitor or a prodrug thereof and an LTB₄ receptor antagonist wherein the LTB₄ receptor antagonist is selected from the group consisting of:
2-[(3S,4R)-3,4-dihydro-4-hydroxy-3-(phenylmethyl)-2H-1-benzopyran-7-yl]-4-(trifluoromethyl)benzoic acid;
4-[[3-[[4-[1-(4-hydroxyphenyl)-1-methylethyl]phenoxy]methyl]phenyl]methoxy]benzenecarboximidamide;
[[4-[[3-[[4-[1-(4-hydroxyphenyl)-1-methyl-ethyl]phenoxy]methyl]-phenyl]methoxy]-phenyl]iminomethyl]carbamic acid, ethyl ester;
4-[1-[4-[[3-[[4-(aminoiminomethyl)phenoxy]-methyl]phenyl]methoxy]phenyl]-1-methylethyl]phenyl beta-D-glucopyranosiduronic acid;
2-[(3R,4S)-3-(1,1′-biphenyl-4-ylmethyl)-4-hydroxy-3,4-dihydro-2H-chromen-7-yl]-4-(trifluoromethyl)benzoic acid;
2-((3S)-3-benzyl-3,4-dihydro-4-hydroxy-2H-chromen-7-yl)-4-chlorobenzoic acid;
2-((3R)-3-benzyl-3,4-dihydro-3,4-dihydroxy-2H-chromen-7-yl)-4-fluorobenzoic acid;
1-carboxyl-1-(((3S,4S)-3,4-dihydroxy-3-(((4-(phenyl)phenyl)-methyl)-chroman-7-yl))cyclopentane;
   a pharmaceutically-acceptable salt thereof; and mixtures of any of these compounds or salts.

Some LTB₄ receptor antagonists useful in the present invention are shown in Table 1. Pharmaceutically-acceptable salts of the compounds listed in Table 1 are also useful in the present invention.

TABLE 1
LTB₄ Receptor Antagonists

| Example | | |
|---|---|---|
| No. | Compound | Reference |
| L-1 | 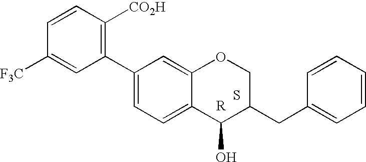 | U.S. Pat. No. 6,436,987 |
| | 2-[(3S,4R)-3,4-dihydro-4-hydroxy-3-(phenylmethyl)-2H-1- | |
| | benzopyran-7-yl]-4-(trifluoromethyl)benzoic acid | |
| L-2 | 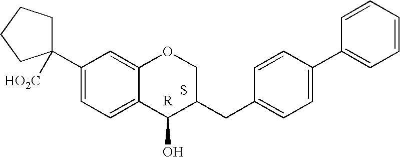 | U.S. Pat. No. 5,550,152 |
| | 1-[3-([1,1′-biphenyl]-4-ylmethyl)-3,4-dihydro-4-hydroxy-2H-1- | |
| | benzopyran-7-yl]-cyclopentanecarboxylic acid, (3S-trans) | |
| L-3 | 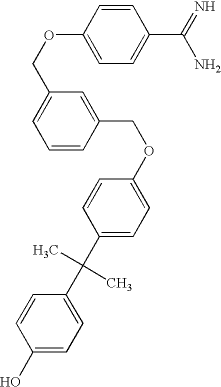 | WO 97/21670 |
| | 4-[[3-[[4-[1-(4-hydroxyphenyl)-1-methylethyl]phenoxy]methyl]- | |
| | phenyl]methoxy]-benzenecarboximidamide | |
| L-4 | 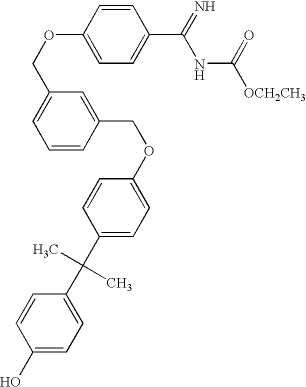 | U.S. Pat. No. 6,417,382 |
| | [[4-[[3-[[4-[1-(4-hydroxyphenyl)-1-methylethyl]phenoxy]methyl]- | |
| | phenyl]methoxy]phenyl]iminomethyl]-carbamic acid, ethyl ester | |
| L-5 | 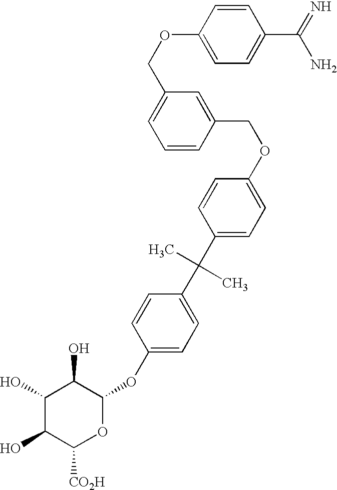 | U.S. Pat. No. 6,197,753 |
| | 4-[1-[4-[[3-[[4-(aminoiminomethyl)- | |
| | phenoxy]-methyl]phenyl]methoxy]phenyl]-1-methylethyl]phenyl | |
| | beta-D-gluco- | |
| | pyranosiduronic acid | |
| L-6 | 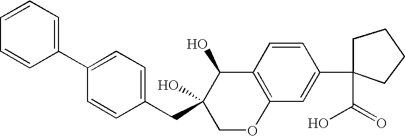 | U.S. Pat. No. 6,122,286 |
| | 1-carbonyl-1-(((3S,4S)-3,4-dihydroxy-3-(((4-(phenyl)phenyl)- | |
| | methyl)-chroman-7-yl))cyclopentane | |
| L-7 | 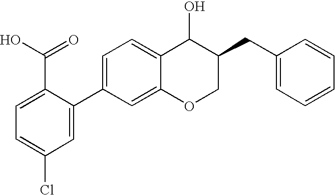 | U.S. Pat. No. 5,552,435 |
| | 2-((3S)-3-benzyl-3,4-dihydro-4-hydroxy- | |
| | 2H-chromen-7-yl)-4-chlorobenzoic acid | |
| L-8 | 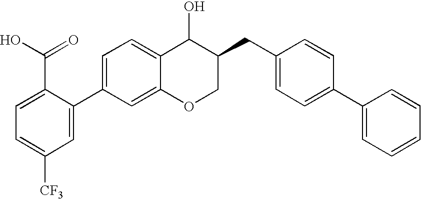 | U.S. Pat. No. 5,552,435 |
| | 2-[(3R,4S)-3-(1,1′-biphenyl-4-ylmethyl)-4-hydroxy-3,4-dihydro- | |
| | 2H-chromen-7-yl]-4-(trifluoromethyl)benzoic acid | |
| L-9 | 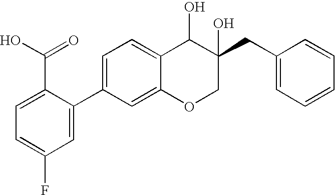 | U.S. Pat. No. 6,051,601 |
| | 2-((3R)-3-benzyl-3,4-dihydro-3,4-dihydroxy- | |
| | 2H-chromen-7-yl)-4-fluorobenzoic acid | |
| L-10 | 29311 Lily LTB₄ receptor antagonist | |

In one embodiment of the present invention the LTB₄ receptor antagonist comprises 2-[(3S,4R)-3,4-dihydro-4-hydroxy-3-(phenylmethyl)-2H-1-benzopyran-7-yl]-4-(trifluoromethyl)benzoic acid, or a salt thereof.

Some compounds which are useful as COX-2 selective inhibitors in the present invention are listed individually in Table 2. These compounds can be used in the present invention singly or in combinations with two or more COX-2 selective inhibitors. Furthermore, pharmaceutically acceptable salts and prodrugs of the compounds listed in Table 2 are also useful in the present invention.

TABLE 2
Some Useful COX-2 Inhibitors

| Compound No. | Compound | Reference |
|---|---|---|
| C-1 | 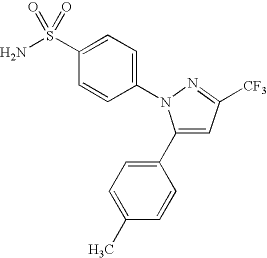 | U.S. Pat. No. 5,466,823 |
| | celecoxib | |
| C-2 | 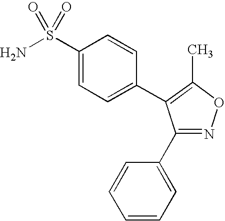 | U.S. Pat. No. 5,633,272 |
| | valdecoxib | |
| C-3 | 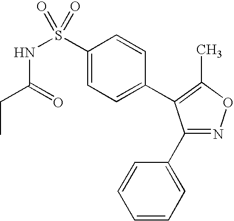 | U.S. Pat. No. 5,932,598 |
| | parecoxib | |
| C-4 | 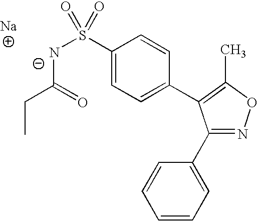 | U.S. Pat. No. 5,932,598 |
| | parecoxib sodium | |
| C-5 | 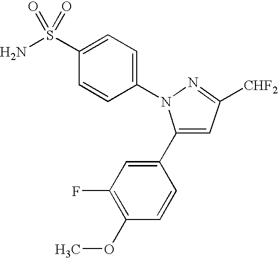 | U.S. Pat. No. 5,521,207 |
| | deracoxib | |
| C-6 | 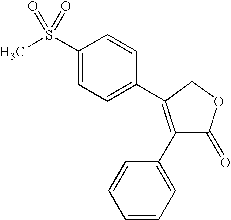 | U.S. Pat. No. 5,474,995 |
| | rofecoxib | |
| C-7 | 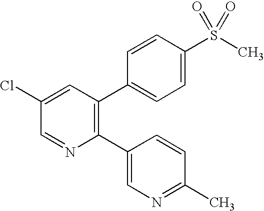 | PCT PatentApplication No.WO 98/03484 |
| | etoricoxib | |
| C-8 | 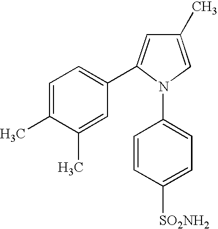 | U.S. Pat. No. 5,908,858 |
| | 4-Methyl-2-(3,4-dimethylphenyl)-1- | |
| | (4-sulfamoyl-phenyl)-1H-pyrrole | |
| C-9 | 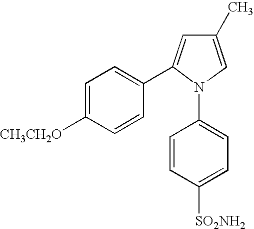 | U.S. Pat. No. 5,908,858 |
| | 2-(4-Ethoxyphenyl)-4-methyl-1- | |
| | (4-sulfamoylphenyl)-1H-pyrrole | |
| C-10 | 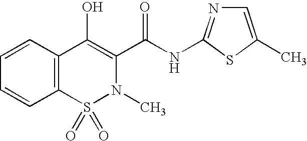 | Reg. No. 71125-38-7 |
| | meloxicam | |
| C-11 | 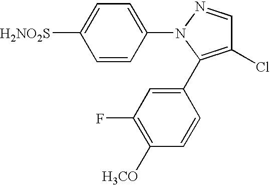 | Reg. No. 265114-23-6 |
| | cimicoxib | |

In one embodiment of the present invention, the therapeutic composition is comprised of any of the LTB₄ receptor antagonists, prodrugs, or salts thereof, of Table 1 and any of the COX-2 inhibitors, prodrugs, or salts thereof, of Table 2.

In one embodiment, the COX-2 selective inhibitor comprises one or more diarylheterocyclic COX-2 selective inhibitors.

Alternatively the COX-2 selective inhibitor can be a chromene COX-2 selective inhibitor. U.S. Pat. No. 6,024,356 (hereby incorporated by reference) describes some useful chromene COX-2 selective inhibitors. Additional useful COX-2 selective inhibitors are described in U.S. patent application Ser. No. 10/801,446, (hereby incorporated by reference). Still further useful COX-2 selective inhibitors are described in U.S. patent application Ser. No. 10/801,429, (hereby incorporated by reference).

In one embodiment, the COX-2 inhibitor comprises celecoxib.

In another embodiment of the present invention the COX-2 selective inhibitor is celecoxib and the LTB₄ receptor antagonist is 2-[(3S,4R)-3,4-dihydro-4-hydroxy-3-(phenylmethyl)-2H-1-benzopyran-7-yl]-4-(trifluoromethyl)benzoic acid (Compound L1). Alternatively, a pharmaceutically acceptable salt of Compound L1 is useful in the present invention. For example, a useful pharmaceutically acceptable salt of Compound L1 is an ethylene diamine salt of L1, for example the mono(ethylene diamine) salt of L1. Such salts and polymorphs of L1 useful in the present invention are described in U.S. Pat. No. 6,436,987, hereby incorporated by reference. In another embodiment of the present invention the COX-2 selective inhibitor is valdecoxib and the LTB₄ receptor antagonist is Compound L1.

Another embodiment of the present invention provides a method for the treatment, prevention, or inhibition of inflammation, an inflammation-related disorder, pain-related disorder, or pain in a subject in need of such prevention, treatment, or inhibition, the method comprising administering to the subject a composition comprising at least one COX-2 selective anti-inflammatory compound, a salt, a prodrug, or mixtures thereof, and at least one LTB₄ receptor antagonist, a salt, a prodrug, or mixtures thereof.

Another embodiment of the present invention provides a method for the treatment, prevention, or inhibition of inflammation, an inflammation-related disorder, pain-related disorder, or pain in a subject in need of such prevention, treatment, or inhibition, the method comprising administering to the subject a composition comprising at least one COX-2 selective inhibitor selected from Table 2 (or salt, prodrug, or mixtures thereof), and at least one LTB₄ receptor antagonist selected from Table 1 (or salt or mixtures thereof). For example the present invention provides a method for the treatment, prevention, or inhibition of inflammation. In another embodiment the present invention provides a method for the treatment, prevention, or inhibition of an inflammation-related disorder.

The present invention further provides a method for the treatment, prevention, or inhibition of inflammation, an inflammation-related disorder, pain-related disorder, or pain in a subject in need of such prevention, treatment, or inhibition, wherein the method comprises administering to the subject a composition comprising at least one COX-2 selective anti-inflammatory compound, salt, prodrug, or mixtures thereof, and at least one LTB₄ receptor antagonist compound, wherein the LTB₄ receptor antagonist compound comprises one or more compounds selected from the group consisting of 2-[(3S,4R)-3,4-dihydro-4-hydroxy-3-(phenylmethyl)-2H-1-benzopyran-7-yl]-4-(trifluoromethyl)benzoic acid; a salt thereof; and mixtures thereof.

Another embodiment of the present invention, is a method for the treatment, prevention, or inhibition of inflammation, an inflammation-related disorder, pain-related disorder, or pain in a subject in need of such prevention, treatment, or inhibition, the method comprising administering to the subject a composition comprising celecoxib, or a salt or prodrug or mixture thereof, and an LTB4 receptor antagonist compound.

Another embodiment of the present invention, is a method for the treatment, prevention, or inhibition of inflammation, an inflammation-related disorder, pain-related disorder, or pain in a subject in need of such prevention, treatment, or inhibition, the method comprising administering to the subject a composition comprising celecoxib, or a salt or prodrug or mixture thereof, and 2-[(3S,4R)-3,4-dihydro-4-hydroxy-3-(phenylmethyl)-2H-1-benzopyran-7-yl]-4-(trifluoromethyl)benzoic acid; or a prodrug or salt or mixture thereof.

In another embodiment, the present invention provides a therapeutic composition comprising a COX-2 selective inhibitor or a prodrug thereof and an LTB₄ receptor antagonist wherein the LTB₄ receptor antagonist comprises 2-[(3S,4R)-3,4-dihydro-4-hydroxy-3-(phenylmethyl)-2H-1-benzopyran-7-yl]-4-(trifluoromethyl)benzoic acid (compound L-1), or one or more salts thereof, or mixtures thereof. Some useful forms of compound L-1 are described in U.S. Pat. No. 6,436,987, herein incorporated by reference. Each compound of Table 1 can be used in its acid or base form (for example conjugate acid or conjugate base) or in the form of any of its pharmaceutically acceptable salts. For example, compound L-1 can be used in the form of any of its pharmaceutically acceptable salts. Such salts include the ethylenediamine salt and crystals of the ethylenediamine salt. Furthermore, each compound of Table 1 can be used in any of its crystalline forms (or mixtures thereof) or in an amorphous form. For example some useful crystalline forms of compound L-1 are described in U.S. Pat. No. 6,435,987.

Compositions of the present invention can be made using any one or more of the above LTB₄ receptor antagonists in combination with any one or more COX-2 selective inhibitors. For example, the compositions of the present invention can be made using any one or more of the above LTB₄ receptor antagonists in combination with any one or more of the above COX-2 selective inhibitors. Table 3 lists some individual combinations of LTB₄ receptor antagonists with COX-2 selective inhibitors which are useful in the present invention.

TABLE 3

| | COX-2 Selective | LTB₄ Receptor |
|---|---|---|
| Example No. | Inhibitor | Antagonist |
| 1 | Celecoxib | L-1 |
| 2 | Celecoxib | L-2 |
| 3 | Celecoxib | L-3 |
| 4 | Celecoxib | L-4 |
| 5 | Celecoxib | L-5 |
| 6 | Celecoxib | L-6 |
| 7 | Celecoxib | L-7 |
| 8 | Celecoxib | L-8 |
| 9 | Celecoxib | L-9 |
| 10 | Valdecoxib | L-1 |
| 11 | Valdecoxib | L-2 |
| 12 | Valdecoxib | L-3 |
| 13 | Valdecoxib | L-4 |
| 14 | Valdecoxib | L-5 |
| 15 | Valdecoxib | L-6 |
| 16 | Valdecoxib | L-7 |
| 17 | Valdecoxib | L-8 |
| 18 | Valdecoxib | L-9 |
| 19 | Parecoxib | L-1 |
| 20 | Parecoxib | L-2 |
| 21 | Parecoxib | L-3 |
| 22 | Parecoxib | L-4 |
| 23 | Parecoxib | L-5 |
| 24 | Parecoxib | L-6 |
| 25 | Parecoxib | L-7 |
| 26 | Parecoxib | L-8 |
| 27 | Parecoxib | L-9 |
| 28 | Deracoxib | L-1 |
| 29 | Deracoxib | L-2 |
| 30 | Deracoxib | L-3 |
| 31 | Deracoxib | L-4 |
| 32 | Deracoxib | L-5 |
| 33 | Deracoxib | L-6 |
| 34 | Deracoxib | L-7 |
| 35 | Deracoxib | L-8 |
| 36 | Deracoxib | L-9 |
| 37 | Rofecoxib | L-1 |
| 38 | Rofecoxib | L-2 |
| 39 | Rofecoxib | L-3 |
| 40 | Rofecoxib | L-4 |
| 41 | Rofecoxib | L-5 |
| 42 | Rofecoxib | L-6 |
| 43 | Rofecoxib | L-7 |
| 44 | Rofecoxib | L-8 |
| 45 | Rofecoxib | L-9 |
| 46 | Etoricoxib | L-1 |
| 47 | Etoricoxib | L-2 |
| 48 | Etoricoxib | L-3 |
| 49 | Etoricoxib | L-4 |
| 50 | Etoricoxib | L-5 |
| 51 | Etoricoxib | L-6 |
| 52 | Etoricoxib | L-7 |
| 53 | Etoricoxib | L-8 |
| 54 | Etoricoxib | L-9 |
| 55 | Lumiracoxib | L-1 |
| 56 | Lumiracoxib | L-2 |
| 57 | Lumiracoxib | L-3 |
| 58 | Lumiracoxib | L-4 |
| 59 | Lumiracoxib | L-5 |
| 60 | Lumiracoxib | L-6 |
| 61 | Lumiracoxib | L7 |
| 62 | Lumiracoxib | L-8 |
| 63 | Lumiracoxib | L--9 |
| 64 | Compound No. C-8 | L-1 |
| 65 | Compound No. C-8 | L2 |
| 66 | Compound No. C-8 | L-3 |
| 67 | Compound No. C-8 | L-4 |
| 68 | Compound No. C-8 | L-5 |
| 69 | Compound No. C-8 | L6 |
| 70 | Compound No. C-8 | L-7 |
| 71 | Compound No. C-8 | L-8 |
| 72 | Compound No. C-8 | L9 |
| 73 | Compound No. C-9 | L-1 |
| 74 | Compound No. C-9 | L-2 |
| 75 | Compound No. C-9 | L-3 |
| 76 | Compound No. C-9 | L-4 |
| 77 | Compound No. C-9 | L-5 |
| 78 | Compound No. C-9 | L-6 |
| 79 | Compound No. C-9 | L-7 |
| 80 | Compound No. C-9 | L-8 |
| 81 | Compound No. C-9 | L-9 |
| 82 | meloxicam | L-1 |
| 83 | meloxicam | L-2 |
| 84 | meloxicam | L-3 |
| 85 | meloxicam | L-4 |
| 86 | meloxicam | L-5 |
| 87 | meloxicam | L-6 |
| 88 | meloxicam | L-7 |
| 89 | meloxicam | L-8 |
| 90 | meloxicam | L-9 |
| 91 | cimicoxib | L-1 |
| 92 | cimicoxib | L-2 |
| 93 | cimicoxib | L-3 |
| 94 | cimicoxib | L-4 |
| 95 | cimicoxib | L-5 |
| 96 | cimicoxib | L-6 |
| 97 | cimicoxib | L-7 |
| 98 | cimicoxib | L-8 |
| 99 | cimicoxib | L-9 |

Pharmaceutically acceptable salts are useful in the compositions of the present invention for a variety of reasons including their aqueous solubility. Such salts must have a pharmaceutically acceptable anion or cation. Illustrative pharmaceutically acceptable salts are prepared from formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, b-hydroxybutyric, galactaric and galacturonic acids.

Suitable pharmaceutically acceptable base addition salts of compounds of the present invention include metallic ion salts and organic ion salts. In some embodiments metallic ion salts include, but are not limited to appropriate alkali metal (group Ia) salts, alkaline earth metal (group IIa) salts and other physiological acceptable metal ions. Such salts can be made from the ions of aluminum, calcium, lithium, magnesium, potassium, sodium and zinc. In some embodiments organic salts can be made from primary, secondary, tertiary amines and quaternary ammonium salts, including in part, trimethylamine, diethylamine, N,N′-dibenzylethylenediamine, chloroprocaine, choline, diethanolamine, ethylenediamine, meglumine (N-methylglucamine) and procaine. All of the above salts can be prepared by those skilled in the art by conventional means from the corresponding compound of the present invention.

Pharmaceutically acceptable cations include metallic ions and organic ions. For example, useful metallic ions include, but are not limited to appropriate alkali metal salts, alkaline earth metal salts and other physiological acceptable metal ions. Exemplary ions include aluminum, calcium, lithium, magnesium, potassium, sodium and zinc in their usual valences. In some embodiments organic ions include protonated primary, secondary, tertiary amines and quaternary ammonium cations, including in part, trimethylamine, diethylamine, N,N′-dibenzylethylenediamine, chloroprocaine, choline, diethanolamine, ethylenediamine, meglumine (N-methylglucamine) and procaine.

Exemplary pharmaceutically acceptable acids include without limitation hydrochloric acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid, oxalic acid, oxalacetic acid, fumaric acid, propionic acid, aspartic acid, glutamic acid, benzoic acid, and the like.

The pharmaceutical composition of the present invention can take a wide variety of forms. For example, the composition can take the form of a tablet, a lozenge, a sachet, a capsule, a chewing gum, a chewable tablet, a controlled release formulation, a sustained-release formulation, a fast-dissolving film, a gel (e.g., a gel capsule), a semi-solid, a solution (aqueous or non-aqueous), a suspension, an intimate mixture of the components, or any combination of two or more of the above.

In one embodiment of the present invention, the composition comprised of a COX-2 inhibitor and an LTB₄ inhibitor is a solid dosage form. For example, the solid dosage form can be an oral dosage form. In yet another embodiment, the oral dosage form is selected from a group consisting of a tablet, a capsule, a suppository, a pill, a gel cap, and granules. In another embodiment the oral dosage form is a capsule. In another embodiment the capsule is a time release capsule. Such a controlled release capsule can, for example, release the active ingredients from a matrix, or in another example it can release the active ingredients at different rates from a mixture of controlled release matrices. In another embodiment, the oral dosage form is a tablet dosage form. In another embodiment the tablet dosage form can be, for example, a multiple layer tablet dosage form (for example, a separate layer for each active ingredient), a wafer, a sustained release tablet dosage form, a core-mantle tablet dosage form, and a side-by-side tablet dosage form (for example, a side for each active ingredient). In another embodiment the tablet dosage form comprises a multiple layer tablet dosage form. In another embodiment the tablet dosage form comprises a side-by-side tablet dosage form. In another embodiment the tablet dosage form comprises a sustained release tablet dosage form. In yet another embodiment, the tablet dosage form comprises a core and mantle tablet dosage form. In yet another embodiment of the present invention, the tablet dosage form comprises an osmotic tablet containing one drug in the core and another drug in the coating. The osmotic tablet can also contain both drugs in the core and another component such as an excipient or other in the coating.

Pharmaceutical compositions suitable for oral administration can be presented in discrete units, such as capsules, sachets, lozenges, or tablets, each containing a predetermined amount of at least one therapeutic compound useful in the present invention; as a powder or granules; as a solution or a suspension in an aqueous or non-aqueous liquid; or as an oil-in-water or water-in-oil emulsion. As indicated, such compositions can be prepared by any suitable method of pharmacy, which includes the step of bringing into association the active compound(s) and the carrier (which can constitute one or more accessory ingredients). In general, the compositions are prepared by uniformly and intimately admixing the active compound with a liquid or finely divided solid carrier, or both, and then, if necessary, shaping the product. For example, a tablet can be prepared by compressing or molding a powder or granules of the compound, optionally with one or more accessory ingredients. Compressed tablets can be prepared by compressing, in a suitable machine, the compound in a free-flowing form, such as a powder or granules optionally mixed with a binder, lubricant, inert diluent and/or surface active/dispersing agent(s). Molded tablets can be made by molding, in a suitable machine, the powdered compound moistened with an inert liquid diluent.

Syrups and elixirs containing a COX-2 selective inhibitor and an LTB₄ receptor antagonist may be formulated with sweetening agents, for example glycerol, sorbitol, or sucrose. Such formulations may also contain a demulcent, a preservative and flavoring and coloring agents. Liquid dosage forms for oral administration can include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, and sweetening, flavoring, and perfuming agents.

Also encompassed by the present invention is buccal or “sub-lingual” administration, which includes lozenges or a chewable gum comprising the compounds, set forth herein. The compounds can be deposited in a flavored base, usually sucrose, and acacia or tragacanth, and pastilles comprising the compounds in an inert base such as gelatin and glycerin or sucrose and acacia.

Pharmaceutical compositions suitable for parenteral administration can conveniently comprise sterile aqueous preparations of a compound of the present invention. These preparations can be, for example, administered intravenously, although administration can also be effected by means of subcutaneous, intramuscular, or intradermal injection or by infusion. Such preparations can conveniently be prepared by admixing the compound with water and rendering the resulting solution sterile and isotonic with the blood. Injectable compositions according to the invention will generally contain from 0.1 to 10% w/w of a compound disclosed herein.

Injectable preparations, for example, sterile injectable aqueous or oleaginous suspensions may be formulated according to the known art using suitable dispersing or setting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed including synthetic mono- or diglycerides. In addition, fatty acids such as oleic acid find use in the preparation of injectables.

Administration of either one or both of the COX-2 inhibitor and the LTB₄ receptor antagonist can also be by inhalation, in the form of aerosols or solutions for nebulizers. Therefore, in one embodiment, the COX-2 inhibitor and LTB₄ receptor antagonist are administered by direct inhalation into the respiratory system of a subject for delivery as a mist or other aerosol or dry powder.

Pharmaceutical compositions suitable for topical application to the skin can, for example, take the form of an ointments, creams, lotions, pastes, gels, sprays, powders, jellies, collyriums, solutions or suspensions, aerosols, or oils.

In another embodiment of the present invention, the combination of a COX-2 inhibitor and an LTB₄ receptor antagonist can be provided in a pharmaceutically acceptable carrier or excipient to form a pharmaceutical composition. Thus, in one embodiment, the present invention encompasses a pharmaceutical composition comprising a COX-2 inhibitor, an LTB₄ receptor antagonist, and a pharmaceutically acceptable carrier. And, in another embodiment, the present invention encompasses a pharmaceutical composition comprising a COX-2 inhibitor, an LTB₄ receptor antagonist, and a pharmaceutically acceptable excipient.

Pharmaceutically acceptable carriers and excipients include, but are not limited to, physiological saline, Ringer's solution, phosphate solution or buffer, buffered saline and other carriers known in the art. Pharmaceutical compositions may also include stabilizers, anti-oxidants, colorants, and diluents. Pharmaceutically acceptable carriers and additives are chosen such that side effects from the pharmaceutical compound are minimized and the performance of the compound is not canceled or inhibited to such an extent that treatment is ineffective.

The carrier should be acceptable in the sense of being compatible with the other ingredients of the composition and not be deleterious to the recipient. The carrier can be a solid or a liquid, or both, and is preferably formulated with the compound as a unit-dose composition, for example, a tablet, which can contain from 0.05% to 95% by weight of the active compound.

Carriers which can be used include petroleum jelly (e.g., Vaseline®), lanolin, polyethylene glycols, alcohols, and combinations of two or more thereof.

Solid dosage forms for the methods of the present invention, which include tablets, capsules, pills, and granules, which can be prepared with coatings and shells, such as enteric coatings and others well known in the art.

Compositions intended for oral use may be prepared according to any method known in the art for the manufacture of pharmaceutical compositions and such compositions may contain one or more agents selected from the group consisting of sweetening agents, flavoring agents, coloring agents, taste masking agents, and preserving agents in order to provide pharmaceutically useful and palatable preparations. Tablets contain the active ingredient in admixture with non-toxic pharmaceutically acceptable excipients, which are suitable for the manufacture of tablets. These excipients may be, for example, inert diluents, such as calcium carbonate, sodium carbonate, lactose, calcium phosphate, sodium phosphate, microcrystalline cellulose, or mannitol, granulating and disintegrating agents, for example, maize starch, or alginic acid, binding agents, for example starch, gelatin, acacia, hydroxypropyl cellulose, hydroxypropyl methylcellulose or polyvinylpyrrolidone, disintegrants, for example, sodium starch glycolate or croscarmellose sodium, and lubricating agents, for example magnesium stearate, stearic acid, or talc. The tablets may also contain glidants such as silicon dioxide and wetting agents like sodium lauryl sulfate. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredients are mixed with an inert solid diluent, for example, lactose, mannitol, microcrystalline cellulose, calcium carbonate, calcium phosphate or kaolin, or as soft gelatin capsules wherein the active ingredients are present as such, or mixed with water or an oil medium, for example, peanut oil, liquid paraffin, soybean oil, olive oil or fractionated coconut oil.

Aqueous suspensions can be produced that contain the active materials in a mixture with excipients suitable for the manufacture of aqueous suspensions. Such excipients are suspending agents, for example, sodium carboxymethylcellulose, methylcellulose, hydroxypropylmethyl-cellulose, sodium alginate, polyvinylpyrrolidone, gum tragacanth, xanthan gum, and gum acacia; dispersing or wetting agents may be naturally-occurring phosphatides, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyoxyethylene sorbitan monooleate.

The aqueous suspensions may also contain one or more preservatives, for example, ethyl or n-propyl p-hydroxybenzoate, one or more coloring agents, one or more flavoring agents, or one or more sweetening agents, such as sucrose or saccharin.

Oily suspensions may be formulated by suspending the active ingredients in an omega-3 fatty acid, a vegetable oil, for example, arachis oil, olive oil, sesame oil or coconut oil, or in a mineral oil such as liquid paraffin. The oily suspensions may contain a thickening agent, for example beeswax, hard paraffin or cetyl alcohol.

Sweetening agents, such as those set forth above, and flavoring agents may be added to provide a palatable oral preparation. These compositions may be preserved by the addition of an antioxidant such as ascorbic acid.

Dispersible powders and granules suitable for preparation of an aqueous suspension by the addition of water provide the active ingredient in admixture with a dispersing or wetting agent, a suspending agent and one or more preservatives. Suitable dispersing or wetting agents and suspending agents are exemplified by those already mentioned above. Additional excipients, for example sweetening, flavoring and coloring agents, may also be present.

The active ingredients may also be administered by injection as a composition wherein, for example, saline, dextrose, or water may be used as a suitable carrier.

Suitable inhalable formulations comprise the active ingredient in a liquid carrier. The carrier is typically water, and most preferably sterile, pyrogen-free water, or a dilute aqueous alcoholic solution, preferably made isotonic, but may be hypertonic with body fluids by the addition of, for example, sodium chloride. Optional additives include preservatives if the formulation is not made sterile, for example, methyl hydroxybenzoate, as well as antioxidants, flavoring agents, volatile oils, buffering agents and surfactants, which are normally used in the preparation of pharmaceutical compositions.

Administration of the compositions of the present invention can also be rectally. These can be prepared by admixing a compound or compounds of the present invention with one or more suitable non-irritating excipients, for example, cocoa butter, synthetic mono- di- or triglycerides, fatty acids and polyethylene glycols that are solid at ordinary temperatures, but liquid at the rectal temperature and will therefore melt in the rectum and release the drug; and then shaping the resulting mixture.

The compositions of the present invention can optionally be supplemented with additional agents such as, for example, viscosity enhancers, preservatives, surfactants and penetration enhancers.

Viscosity is an important attribute of many medications. Drops that have a high viscosity tend to stay in the body for longer periods and thus, increase absorption of the active compounds by the target tissues or increase the retention time. Such viscosity-building agents include, for example, polyvinyl alcohol, polyvinyl pyrrolidone, methylcellulose, hydroxy propyl methylcellulose, hydroxyethyl cellulose, carboxymethyl cellulose, hydroxy propyl cellulose or other agents know to those skilled in the art. Such agents are typically employed at a level of from 0.01% to 2% by weight.

Preservatives are optionally employed to prevent microbial contamination during use. Suitable preservatives include polyquaternium-1, benzalkonium chloride, thimerosal, chlorobutanol, methyl paraben, propyl paraben, phenylethyl alcohol, edetate disodium, sorbic acid, or other agents known to those skilled in the art. The use of polyquaternium-1 as the antimicrobial preservative is preferred. Typically, such preservatives are employed at a level of from 0.001% to 1.0% by weight.

The solubility of the components of the present compositions may be enhanced by a surfactant or other appropriate co-solvent in the composition. Such co-solvents include polysorbate 20, 60, and 80, polyoxyethylene/poly-oxypropylene surfactants (e.g. Pluronic F-68, F-84 and P-103), cyclodextrin, or other agents known to those skilled in the art. Typically, such co-solvents are employed at a level of from 0.01% to 2% by weight.

A penetration enhancer is an agent used to increase the permeability of the skin to an active agent to increase the rate at which the drug diffuses through the skin and enters the tissues and bloodstream. Thus, in one embodiment of the present invention, a penetration enhancer may be added to a COX-2 inhibitor and LTB₄ receptor antagonist topical composition.

Examples of penetration enhancers suitable for use with the compositions of the present invention include: alcohols, such as ethanol and isopropanol; polyols, such as n-alkanols, limonene, terpenes, dioxolane, propylene glycol, ethylene glycol, other glycols, and glycerol; sulfoxides, such as dimethylsulfoxide (DMSO), dimethylformamide, methyl dodecyl sulfoxide, dimethylacetamide; esters, such as isopropyl myristate/palmitate, ethyl acetate, butyl acetate, methyl proprionate, and capric/caprylic triglycerides; ketones; amides, such as acetamides; oleates, such as triolein; various surfactants, such as sodium lauryl sulfate; various alkanoic acids, such as caprylic acid; lactam compounds, such as azone; alkanols, such as oleyl alcohol; dialkylamino acetates, and admixtures thereof.

Pharmaceutically acceptable excipients and carriers encompass all the foregoing and the like. The above considerations concerning effective formulations and administration procedures are well known in the art and are described in standard textbooks.

The dosage regimen to prevent, give relief from, or ameliorate a disease condition described herein is selected in accordance with a variety of factors. These include the type, age, weight, sex, diet, and medical condition of the patient, the severity of the disease, the route of administration, pharmacological considerations such as the activity, efficacy, pharmacokinetics and toxicology profiles of the particular compound employed, whether a drug delivery system is utilized, and whether the compound is administered as part of a drug combination. Thus, the dosage regimen actually employed may vary widely and therefore deviate from the preferred dosage regimen set forth above.

The amount of the present composition that is required to achieve the desired biological effect will, of course, depend on a number of factors such as the specific composition chosen, the use for which it is intended, the mode of administration, and the subject to be treated and the clinical condition of the recipient. Initial treatment of a patient suffering from a therapeutic condition can begin with the dosages indicated above. Treatment should generally be continued as necessary over a period of several weeks to several months or years until the disease condition has been controlled or eliminated. Patients undergoing treatment with the compounds or compositions disclosed herein can be routinely monitored to determine the effectiveness of therapy. Continuous analysis of such data permits modification of the treatment regimen during therapy so that optimal effective amounts of compounds of the present invention are administered at any point in time, and so that the duration of treatment can be determined as well. In this way, the treatment regimen/dosing schedule can be rationally modified over the course of therapy so that the lowest amount of LTB₄ receptor antagonist and the COX-2 selective inhibitor which exhibits satisfactory effectiveness is administered, and so that administration is continued only so long as is necessary to successfully treat the condition.

Pharmaceutical compositions according to the present invention include those suitable for oral, rectal, topical, buccal (e.g., sublingual), and parenteral (e.g., subcutaneous, intramuscular, intradermal, intrathecal, intramedullary, or intravenous) administration, although the most suitable route in any given case will depend on the nature and severity of the condition being treated and on the nature of the particular compound which is being used. In most cases, the route of administration will be oral.

In another embodiment, the present invention provides a kit comprising a container wherein is located the composition of the present invention.

Dosing for oral administration may be with a regimen calling for single daily dose, or for a single dose every other day, or for multiple, spaced doses throughout the day. For oral administration, the pharmaceutical composition may be in the form of, for example, a tablet, capsule, suspension, or liquid. Capsules, tablets, etc., can be prepared by conventional methods well known in the art. The pharmaceutical composition is preferably made in the form of a dosage unit containing a particular amount of the active ingredient or ingredients. Examples of dosage units are tablets or capsules, and may contain one or more therapeutic compounds in an amount described herein.

The compositions of the present invention can also be administered enterally, by inhalation spray, rectally, topically, buccally or parenterally in dosage unit formulations containing conventional nontoxic pharmaceutically acceptable carriers, adjuvants, and vehicles as desired. Parenteral administration includes subcutaneous, intramuscular, intradermal, intramammary, intravenous, and other administrative methods known in the art. Enteral administration includes solution, tablets, sustained release capsules, enteric-coated capsules, and syrups. When administered, the pharmaceutical composition may conveniently be at or near body temperature.

Oral delivery of the combinations of the present invention can include formulations, as are well known in the art, to provide prolonged or sustained delivery of the drug to the gastrointestinal tract by any number of mechanisms. These include, but are not limited to, pH sensitive release from the dosage form based on the changing pH of the small intestine, slow erosion of a tablet or capsule, retention in the stomach based on the physical properties of the formulation, bioadhesion of the dosage form to the mucosal lining of the intestinal tract, or enzymatic release of the active drug from the dosage form. For some of the therapeutic compounds useful in the methods, combinations and compositions of the present invention the intended effect is to extend the time period over which the active drug molecule is delivered to the site of action by manipulation of the dosage form. Thus, enteric-coated and enteric-coated controlled release formulations are within the scope of the present invention. Suitable enteric coatings include cellulose acetate phthalate, polyvinylacetate phthalate, hydroxypropylmethylcellulose phthalate and anionic polymers of methacrylic acid and methacrylic acid methyl ester.

Pharmaceutical compositions suitable for parenteral administration can conveniently comprise sterile aqueous preparations of a compound of the present invention. These preparations are preferably administered intravenously, although administration can also be effected by means of subcutaneous, intramuscular, or intradermal injection or by infusion. Such preparations can conveniently be prepared by admixing the compound with water and rendering the resulting solution sterile and isotonic with the blood. Injectable compositions according to the invention will generally contain from 0.1 to 10% w/w of a compound disclosed herein.

Administration may also be by transvaginal delivery through the use of an intravaginal device. Transvaginal delivery may be desirable for many certain subjects because 10 to 30 times more treatment agent can be delivered transvaginally as can be delivered orally due to the absorption from the vagina, which far exceeds the absorption of drugs from the gastrointestinal tract. Further, vaginal administration generally avoids major problems connected with oral administration, such as gastric and esophageal reflux and ulceration.

Transdermal administration is also possible. Pharmaceutical compositions suitable for transdermal administration can be presented as discrete patches adapted to remain in intimate contact with the epidermis of the recipient for a prolonged period of time. Such patches suitably contain a compound or compounds of the present invention in an optionally buffered, aqueous solution, dissolved and/or dispersed in an adhesive, or dispersed in a polymer. A suitable concentration of the active compound or compounds is about 1% to 35%, preferably about 3% to 15%. As one particular possibility, the compound or compounds can be delivered from the patch by electrotransport or iontophoresis, for example, as described in Pharmaceutical Research 3(6):318 (1986).

The method of the present invention is useful for, but not limited to, the prevention and/or treatment of pain or inflammation or inflammation-related disorders. For example in some embodiments, the inflammation-related disorder is arthritis. In another example, the compounds described herein would be useful for the treatment of pain or inflammation or any inflammation-related disorder described below, such as an analgesic in the treatment of pain and headaches, or as an antipyretic for the treatment of fever. The compounds described herein would also be useful for the treatment of an inflammation-related disorder in a subject suffering from such an inflammation-associated disorder.

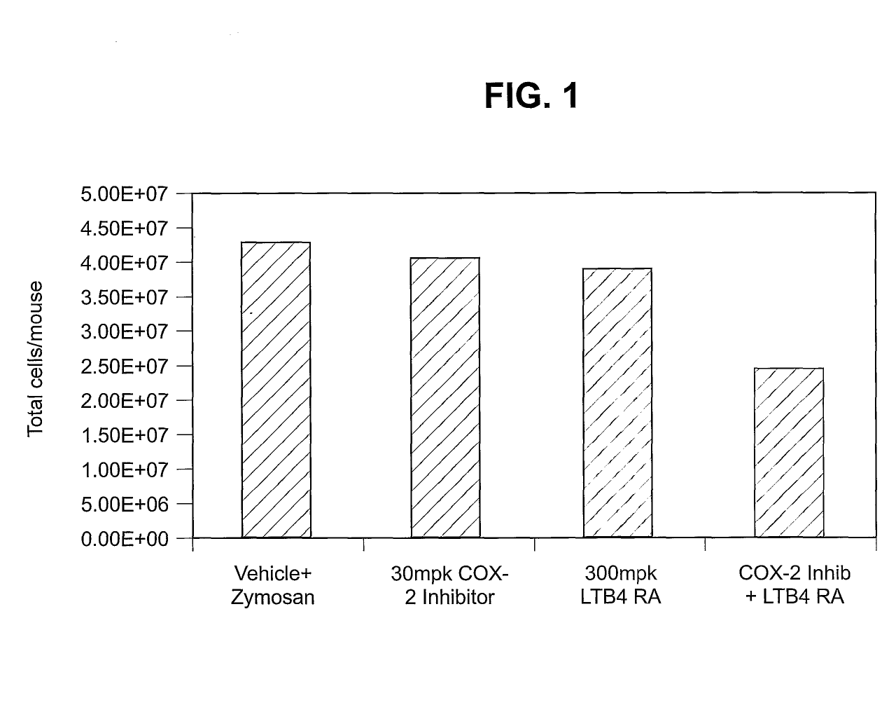

In the present invention, the anti-inflammatory effects of COX-2 inhibitors and LTB₄ inhibitors, both alone and together, can be evaluated using the mouse air pouch model (see Example 1 and FIG. 1). Following the addition of a COX-2 inhibitor, or an LTB₄ inhibitor, or both, to a zymosan-stimulated air pouch, results may be reported as the number of cells that have infiltrated per pouch as an indicator of inflammation.

In some embodiments, the methods and compositions of the present invention encompass the prevention and/or treatment of inflammation-related disorders. In other embodiments, the methods and compositions of the present invention encompass the prevention and/or treatment of any one or more of the disorders selected from the group consisting of connective tissue and joint disorders, pain and pain-related disorders, neoplasia disorders, cardiovascular disorders, otic disorders, ophthalmic disorders, respiratory disorders, gastrointestinal disorders, angiogenesis-related disorders, immunological disorders, allergic disorders, nutritional disorders, infectious diseases and disorders, endocrine disorders, metabolic disorders, neurological and neurodegenerative disorders, psychiatric disorders, hepatic and biliary disorders, musculoskeletal disorders, genitourinary disorders, gynecologic and obstetric disorders, injury and trauma disorders, surgical disorders, dental and oral disorders, sexual dysfunction disorders, dermatologic disorders, hematological disorders, and poisoning disorders.

Also encompassed by the present invention is the treatment of benign, premalignant, metastatic, or malignant neoplasias.

Compositions of the invention will be useful for the prevention or treatment of benign and malignant tumors or neoplasia including cancer, such as colorectal cancer, brain cancer, bone cancer, epithelial cell derived neoplasia (epithelial carcinoma) such as basal cell carcinoma, adenocarcinoma, gastrointestinal cancer such as lip cancer, mouth cancer, esophogeal cancer, small bowel cancer and stomach cancer, colon cancer, liver cancer, bladder cancer, pancreas cancer, ovary cancer, cervical cancer, lung cancer, breast cancer and skin cancer, such as squamus cell and basal cell cancers, prostate cancer, renal cell carcinoma, and other known cancers that effect epithelial cells throughout the body. In one embodiment, the neoplasia is selected from gastrointestinal cancer, Barrett's esophagus, liver cancer, bladder cancer, pancreas cancer, ovary cancer, prostate cancer, cervical cancer, lung cancer, breast cancer and skin cancer, such as squamus cell and basal cell cancers. The compounds can also be used to treat the fibrosis, which occurs with radiation therapy. The method can be used to treat subjects having adenomatous polyps, including those with sporadic adenomatous polyposis (SAP) or familial adenomatous polyposis (FAP). Additionally, the method can be used to prevent polyps from forming in patients at risk of FAP.

In other embodiments, the methods and compositions of the present invention encompass the prevention and treatment of the neoplasia disorders selected from the group consisting of acral lentiginous melanoma, actinic keratoses, adenocarcinoma, adenoid cycstic carcinoma, adenomas, familial adenomatous polyposis, familial polyps, colon polyps, polyps, adenosarcoma, adenosquamous carcinoma, adrenocortical carcinoma, AIDS-related lymphoma, anal cancer, astrocytic tumors, bartholin gland carcinoma, basal cell carcinoma, bile duct cancer, bladder cancer, brain stem glioma, brain tumors, breast cancer, bronchial gland carcinomas, capillary carcinoma, carcinoids, carcinoma, carcinosarcoma, cavernous, central nervous system lymphoma, cerebral astrocytoma, cholangiocarcinoma, chondosarcoma, choriod plexus papilloma/carcinoma, clear cell carcinoma, skin cancer, brain cancer, colon cancer, colorectal cancer, cutaneous T-cell lymphoma, cystadenoma, endodermal sinus tumor, endometrial hyperplasia, endometrial stromal sarcoma, endometrioid adenocarcinoma, ependymal, epitheloid, esophageal cancer, Ewing's sarcoma, extragonadal germ cell tumor, fibrolamellar, focal nodular hyperplasia, gallbladder cancer, gastrinoma, germ cell tumors, gestational trophoblastic tumor, glioblastoma, glioma, glucagonoma, hemangiblastomas, hemangioendothelioma, hemangiomas, hepatic adenoma, hepatic adenomatosis, hepatocellular carcinoma, Hodgkin's lymphoma, hypopharyngeal cancer, hypothalamic and visual pathway glioma, insulinoma, intaepithelial neoplasia, interepithelial squamous cell neoplasia, intraocular melanoma, invasive squamous cell carcinoma, large cell carcinoma, islet cell carcinoma, Kaposi's sarcoma, kidney cancer, laryngeal cancer, leiomyosarcoma, lentigo maligna melanomas, leukemia-related disorders, lip and oral cavity cancer, liver cancer, lung cancer, lymphoma, malignant mesothelial tumors, malignant thymoma, medulloblastoma, medulloepithelioma, melanoma, meningeal, merkel cell carcinoma, mesothelial, metastatic carcinoma, mucoepidermoid carcinoma, multiple myeloma/plasma cell neoplasm, mycosis fungoides, myelodysplastic syndrome, myeloproliferative disorders, nasal cavity and paranasal sinus cancer, nasopharyngeal cancer, neuroblastoma, neuroepithelial adenocarcinoma nodular melanoma, non-Hodgkin's lymphoma, oat cell carcinoma, oligodendroglial, oral cancer, oropharyngeal cancer, osteosarcoma, pancreatic polypeptide, ovarian cancer, ovarian germ cell tumor, pancreatic cancer, papillary serous adenocarcinoma, pineal cell, pituitary tumors, plasmacytoma, pseudosarcoma, pulmonary blastoma, parathyroid cancer, penile cancer, pheochromocytoma, pineal and supratentorial primitive neuroectodermal tumors, pituitary tumor, plasma cell neoplasm, pleuropulmonary blastoma, prostate cancer, rectal cancer, renal cell carcinoma, retinoblastoma, rhabdomyosarcoma, sarcoma, serous carcinoma, small cell carcinoma, small intestine cancer, soft tissue carcinomas, somatostatin-secreting tumor, squamous carcinoma, squamous cell carcinoma, submesothelial, superficial spreading melanoma, supratentorial primitive neuroectodermal tumors, thyroid cancer, undifferentiatied carcinoma, urethral cancer, uterine sarcoma, uveal melanoma, verrucous carcinoma, vaginal cancer, vipoma, vulvar cancer, Waldenstrom's macroglobulinemia, well differentiated carcinoma, and Wilm's tumor.

In another embodiment, the compositions of the present invention are useful for the treatment of the signs and symptoms of cancer treatment. For example the present compositions are useful for the treatment of chemotherapy- or radiation-induced cachexia.

In still other embodiments, the methods and compositions of the present invention encompass the prevention and treatment of the connective tissue and joint disorders selected from the group consisting of arthritis, rheumatoid arthritis, spondyloarthopathies, gouty arthritis, lumbar spondylarthrosis, carpal tunnel syndrome, canine hip dysplasia, systemic lupus erythematosus, juvenile arthritis, osteoarthritis, tendonitis and bursitis.

In other embodiments, the methods and compositions of the present invention encompass the prevention and treatment of the cardiovascular disorders selected from the group consisting of myocardial ischemia, hypertension, hypotension, heart arrhythmias, pulmonary hypertension, hypokalemia, vascular diseases, vascular rejection, atherosclerosis including cardiac transplant atherosclerosis, myocardial infarction, embolism, stroke, thrombosis, including venous thrombosis, angina including unstable angina, coronary plaque inflammation, cardiac ischemia, myocardial infarction, cardiac remodeling, cardiac fibrosis, myocardial necrosis, aneurysm, arterial fibrosis, embolism, vascular plaque inflammation, vascular plaque rupture, bacterial-induced inflammation and viral induced inflammation, edema, swelling, fluid accumulation, cirrhosis of the liver, Bartter's syndrome, myocarditis, arteriosclerosis, atherosclerosis, calcification (such as vascular calcification and valvar calcification), coronary artery disease, heart failure, congestive heart failure, shock, arrhythmia, left ventricular hypertrophy, angina, diabetic nephropathy, kidney failure, eye damage, migraine headaches, aplastic anemia, cardiac damage, diabetic cardiac myopathy, renal insufficiency, renal injury, renal arteriopathy, peripheral vascular disease, cognitive dysfunction, stroke, headache, and inflammation associated with surgical procedures such as vascular grafting including coronary artery bypass surgery, revascularization procedures including angioplasty, stent placement, endarterectomy, or other invasive procedures involving arteries, veins and capillaries.

In other embodiments, the methods and compositions of the present invention encompass the prevention and treatment of the metabolic disorders selected from the group consisting of obesity, overweight, type I and type II diabetes, hypothyroidism, and hyperthyroidism.

In other embodiments, the methods and compositions of the present invention encompass the prevention and treatment of the respiratory disorders selected from the group consisting of asthma, bronchitis, chronic obstructive pulmonary disease (COPD), cystic fibrosis, pulmonary edema, pulmonary embolism, pneumonia, pulmonary sarcoisosis, silicosis, pulmonary fibrosis, respiratory failure, acute respiratory distress syndrome and emphysema.

In other embodiments, the methods and compositions of the present invention encompass the prevention and treatment of the angiogenesis-related disorders selected from the group consisting of angiofibroma, neovascular glaucoma, arteriovenous malformations, arthritis, osler-weber syndrome, atherosclerotic plaques, psoriasis, corneal graft neovascularization, pyogenic granuloma, delayed wound healing, retrolental fibroplasias, diabetic retinopathy, scleroderma, granulations, solid tumors, hemangioma, trachoma, hemophilic joints, vascular adhesions, hypertrophic scars, age-related macular degeneration, coronary artery disease, stroke, cancer, AIDS complications, ulcers and infertility.

In other embodiments, the methods and compositions of the present invention encompass the prevention and treatment of the infectious diseases and disorders selected from the group consisting of viral infections, bacterial infections, prion infections, spirochetes infections, mycobacterial infections, rickettsial infections, chlamydial infections, parasitic infections and fungal infections.

In still further embodiments, the methods and compositions of the present invention encompass the prevention and treatment of the infectious diseases and disorders selected from the group consisting of hepatitis, HIV (AIDS), small pox, chicken pox, common cold, bacterial influenza, viral influenza, warts, oral herpes, genital herpes, herpes simplex infections, herpes zoster, bovine spongiform encephalopathy, septicemia, streptococcus infections, staphylococcus infections, anthrax, severe acquired respiratory syndrome (SARS), malaria, African sleeping sickness, yellow fever, chlamydia, botulism, canine heartworm, rocky mountain spotted fever, lyme disease, cholera, syphilis, gonorrhea, encephalitis, pneumonia, conjunctivitis, yeast infections, rabies, dengue fever, Ebola, measles, mumps, rubella, West Nile virus, meningitis, gastroenteritis, tuberculosis, hepatitis, and scarlet fever.

The present invention also provides a therapy comprising a COX-2 inhibitor in combination with an LTB₄ receptor antagonist, which encompasses the treatment and prevention of such neurodegenerative disorder symptoms as, for example, dementia, aphasia, memory loss, depression, apraxia, anxiety, personality disorders, agnosia, and hallucinations in a subject suffering from such symptoms.

As used herein, the terms “neurodegenerative disorder” is defined as having any abnormality of one or more nerves, a post-surgical condition of any tissue that is comprised of nerves, or an age-related condition of one or more nerves. As used herein, the term “neuro” or “nerve” includes any component or structure found within or on the central nervous system or peripheral nervous system, including, but not limited to, neurons, brain tissue, spinal cord tissue, glial cells, astrocytes, dendrites, cholinergic receptors, adrenergic receptors, gaba receptors, serotoninergic (5-HT) receptors, glutamate receptors, endorphin-enkephalin (opioid) receptors, Schwann cells, axons, oligodendrocytes, microglia, ependyma, myelin sheaths, and any other neurological tissue within a subject's body.

The terms “neurodegenerative disorder” also include any complications that arise from having such a disorder. For example, many chronic neurodegenerative disorders are often associated with complications, such as, for example, complications caused by immobility, muscle contractures, reduced life span, opportunistic infections, and pressure sores, any of which may eventually arise from having a chronic or recurring neurodegenerative disorder. Behavioral neurodegenerative disorder complications include hostility, aggression, agitation, wandering, and uncooperativeness. Psychiatric complications include depression, anxiety, paranoid reactions, delusions, and hallucinations.

Neurodegenerative disorders may arise in a subject via several determinants including chronic substance abuse, vascular disease, and inadequate consumption of vitamins, infectious agents, causative agents, brain cancer, mental or physical trauma, brain trauma and genetics. The methods and compositions of the present invention are intended to treat a subject suffering from a neurodegenerative disorder regardless of how the disorder first arose.

In one embodiment, the methods and compositions of the present invention encompass the prevention and treatment of the neurodegenerative disorders selected from the group consisting of cortical dementias, general dementia, old-age, Alzheimer's disease, vascular dementia, multi-infarct dementia, pre-senile dementia, alcoholic dementia, senile dementias, stroke, coma, seizures, epilepsy, amnesia, hypovolemic shock, phenylketonuria, aminoacidurias, Tay-Sachs, Niemann-Pick, Gaucher's diseases, Hurler's syndrome, Krabbe's disease, leukodystrophies, traumatic shock, reperfusion injury, multiple sclerosis, AIDS associated dementia, neuron toxicity, head trauma, adult respiratory disease (ARDS), acute spiral cord injury, Parkinson's Disease, frontotemporal dementia, Pick's disease, ischemia, palsy, supranuclear palsy, corticobasal degeneration, multi-infarct dementia, Creutzfeldt-Jakob disease, normal pressure hydrocephalus, delirium, headaches, migraine headaches, Parkinson's disease, memory loss, senility, amyotrophy, ALS, muscular dystrophies, epilepsy, schizophrenia, depression, anxiety, anxiety, autism, phobias, spongiform encephalopathies, Huntington's Chorea, ischemia, obsessive-compulsive disorder, anxiety-related disorders, stress-related disorders, psychosis, neuroendocrine system disorders, thermoregulation disorders, vasoreactive headaches, sexual dysfunction, tooth-germ morphogenesis disorders, Tourette's syndrome, autism, attention deficit disorders, hyperactivity disorders, sleep disorders, social phobias, urinary incontinence, vasospasm, stroke, eating disorders such as obesity, anorexia and bullemia, manic depression, bipolar disorders, drug addiction, alcoholism and smoking addiction. In addition, the neurodegenerative disorders that may be treated with the compositions and methods described herein, include a subject who is otherwise normal, but wishes to improve upon certain cognitive abilities, such as memory retention and thought processes.

In other embodiments, the methods and compositions of the present invention encompass the prevention and treatment of the dermatological disorders selected from the group consisting of acne, psoriasis, eczema, burns, poison ivy, poison oak and dermatitis.

In other embodiments, the methods and compositions of the present invention encompass the prevention and treatment of the surgical disorders selected from the group consisting of pain and swelling following surgery, infection following surgery and inflammation following surgery.

In other embodiments, the methods and compositions of the present invention encompass the prevention and treatment of the gastrointestinal disorders selected from the group consisting of inflammatory bowel disease, irritable bowel syndrome, Crohn's disease, gastritis, irritable bowel syndrome, diarrhea, constipation, dysentery, ulcerative colitis, gastric esophageal reflux, gastric ulcers, gastric varices, ulcers, and heartburn.

In other embodiments, the methods and compositions of the present invention encompass the prevention and treatment of the otic disorders selected from the group consisting of otic pain, inflammation, otorrhea, otalgia, fever, otic bleeding, Lermoyez's syndrome, Meniere's disease, vestibular neuronitis, benign paroxysmal positional vertigo, herpes zoster oticus, Ramsay Hunt's syndrome, viral neuronitis, ganglionitis, geniculate herpes, labyrinthitis, purulent labyrinthitis, viral endolymphatic labyrinthitis, perilymph fistulas, noise-induced hearing loss, presbycusis, drug-induced ototoxicity, acoustic neuromas, aerotitis media, infectious myringitis, bullous myringitis, otitis media, otitis media with effusion, acute otitis media, secretory otitis media, serous otitis media, acute mastoiditis, chronic otitis media, otitis extema, otosclerosis, squamous cell carcinoma, basal cell carcinoma, nonchromaffin paragangliomas, chemodectomas, globus jugulare tumors, globus tympanicum tumors, external otitis, perichondritis, aural eczematoid dermatitis, malignant external otitis, subperichondrial hematoma, ceruminomas, impacted cerumen, sebaceous cysts, osteomas, keloids, otalgia, tinnitus, vertigo, tympanic membrane infection, typanitis, otic furuncles, otorrhea, acute mastoiditis, petrositis, conductive and sensorineural hearing loss, epidural abscess, lateral sinus thrombosis, subdural empyema, otitic hydrocephalus, Dandy's syndrome, bullous myringitis, cerumen-impacted, diffuse external otitis, foreign bodies, keratosis obturans, otic neoplasm, otomycosis, trauma, acute barotitis media, acute eustachian tube obstruction, post-otic surgery, postsurgical otalgia, cholesteatoma, conductive and sensorineural hearing loss, epidural abscess, lateral sinus thrombosis, subdural empyema and otitic hydrocephalus.

In other embodiments, the methods and compositions of the present invention encompass the prevention and treatment of the ophthalmic disorders selected from the group consisting of retinopathies, uveitis, ocular photophobia, acute injury to the eye tissue, conjunctivitis, age-related macular degeneration diabetic retinopathy, detached retina, glaucoma, vitelliform macular dystrophy type 2, gyrate atrophy of the choroid and retina, conjunctivitis, corneal infection, Fuchs' dystrophy, iridocorneal endothelial syndrome, retinitis, keratoconus, lattice dystrophy, map-dot-fingerprint dystrophy, ocular herpes, pterygium, myopia, hyperopia, and cataracts.

The combinations and methods would also be useful in the treatment of pain, but not limited to postoperative pain, dental pain, muscular pain, neuropathic pain and pain resulting from cancer.

In other embodiments, the methods and compositions of the present invention encompass the prevention and treatment of menstrual cramps, kidney stones, minor injuries, wound healing, vaginitis, candidiasis, sinus headaches, tension headaches, periarteritis nodosa, thyroiditis, myasthenia gravis, sarcoidosis, nephrotic syndrome, Bahcet's syndrome, polymyositis, gingivitis, hypersensitivity, swelling occurring after injury, closed head injury, liver disease, and endometriosis.

The methods and compositions of the present invention not only encompass the prevention or treatment of pain or inflammation or inflammation-related disorders in humans, but also in several animals. For example, many animals also suffer adverse consequences related to pain or inflammation inflammation-related disorders. Moreover, many inflammation-related disorders in dogs respond to the same treatment used in humans. Accordingly, besides being useful for humans, the methods and compositions of the present invention also encompass the treatment and prevention of pain or inflammation, and in some embodiments, inflammation-related disorders, in other mammals, including horses, dogs, cats, sheep, pigs, cattle, and the like. Thus, it is preferred that the subject is an animal, and yet more preferred, the subject is a mammal. Preferably, the mammal is a human.

It will be appreciated that the amount of the present composition required for use in the treatment or prevention of the conditions described herein will vary within wide limits and will be adjusted to the individual requirements in each particular case. In general, for administration to adults, an appropriate daily dosage is described herein, although the dosages that are identified herein may be exceeded if expedient. The daily dosage can be administered as a single dosage or in divided dosages.

The appropriate dosage level of a COX-2 inhibitor will generally be from about 0.01 mg per kg to about 140 mg per kg subject body weight per day, which may be administered in single or multiple doses. In one embodiment, the dosage level will be about 0.1 mg/kg to about 25 mg/kg per day and in another embodiment about 0.5 mg/kg to about 10 mg/kg per day.

In larger mammals, for example humans, a typical indicated dose is about 0.5 mg to 7 grams orally per day. A compound may be administered on a regimen of several times per day, for example 1 to 4 times per day, alternatively once or twice per day.

The amount of the COX-2 inhibitor that may be combined with the carrier materials to produce a single dosage form will vary depending upon the host treated and the particular mode of administration. For example, a formulation intended for the oral administration of humans may contain from 0.5 mg to 7 g of active agent compounded optionally with an appropriate and convenient amount of carrier material, which may vary from about 5 to about 95 percent of the total composition. Dosage unit forms for the COX-2 inhibitor will generally contain between from about 1 mg to about 500 mg of an active ingredient, typically 25 mg, 50 mg, 100 mg, 200 mg, 300 mg, 400 mg, 500 mg, 600 mg, 800 mg, or 1000 mg.

A total daily dose of a COX-2 inhibitor can generally be in the range of from about 0.001 to about 10,000 mg/day in single or divided doses, in one embodiment about 1.0 mg to about 2,000 mg. It is understood, however, that specific dose levels of the therapeutic agents or therapeutic approaches of the present invention for any particular patient depends upon a variety of factors including the activity of the specific compound employed, the age, body weight, general health, sex, and diet of the patient, the time of administration, the rate of excretion, the drug combination, and the severity of the particular disease being treated and form of administration.

In one embodiment of the present invention, the dosages of the LTB₄ component ranges from about 0.01 mg to about 5,000 mg or any other dose, dependent upon the specific modulator.

In one embodiment of the present invention, the ratio of a COX-2 inhibitor to an LTB₄ receptor antagonist is 1:1. In another embodiment of the present invention, the ratio of a COX-2 inhibitor to an LTB₄ receptor antagonist is one, to any concentration greater than one. For example, in one embodiment, the ratio of COX-2 inhibitor to an LTB₄ receptor antagonist is 1:2. In another embodiment of the present invention, the ratio of a COX-2 inhibitor to an LTB₄ receptor antagonist is any concentration greater than one, to one. For example, in one embodiment of the present invention, the ratio of COX-2 inhibitor to an LTB₄ receptor antagonist is 2:1. For example, in one embodiment, the composition comprises about 200 mg celecoxib and about 200 mg of an LTB₄ receptor antagonist. In another embodiment of the present invention, the composition comprises about 200 mg celecoxib and about 400 mg of an LTB₄ receptor antagonist and in yet another embodiment of the invention, the composition comprises about 100 mg celecoxib and about 200 mg of an LTB₄ receptor antagonist.

Treatment dosages generally may be titrated to optimize safety and efficacy. Typically, dosage-effect relationships from in vitro initially can provide useful guidance on the proper doses for patient administration. Studies in animal models also generally may be used for guidance regarding effective dosages for treatment of pain or inflammation in accordance with the present invention. In terms of treatment protocols, it should be appreciated that the dosage to be administered will depend on several factors, including the particular agent that is administered, the route administered, the condition of the particular patient, etc. Generally speaking, one will desire to administer an amount of the compound that is effective to achieve a serum level commensurate with the concentrations found to be effective in vitro. Thus, where a compound is found to demonstrate in vitro activity at, e.g., 10 micromolar, one will desire to administer an amount of the drug that is effective to provide about a 10 micromolar concentration in vivo. Determination of these parameters is well within the skill of the art.

Dosages for the combination therapy provided herein may be determined and adjusted based on the efficacy demonstrated in reducing or preventing the symptoms of pain or inflammation or inflammation-related disorders. In addition, one of ordinary skill in the art will know how to measure and quantify the presence or absence of pain or inflammation symptoms.

### c. DETAILED EXAMPLES

The starting materials for use in the compositions and methods of the invention are known or can be prepared by conventional methods known to a skilled person or in an analogous manner to processes described in the art.

Generally, the methods of the present invention can be performed as follows.

Herein, compounds used in the following examples are referred to by their example number (see Tables 1 and 2). For instance, LTB₄ compound L-1 found in Table 1 is 2-[(3S,4R)-3,4-dihydro-4-hydroxy-3-(phenylmethyl)-2H-1-benzopyran-7-yl]-4-(trifluoromethyl)benzoic acid. COX-2 inhibitors such as C-1 (celecoxib) are found in Table 2.

### Example 1

### COX-2 Inhibitor Activity and In Vitro COX-1 Selectivity

The in vitro COX-2 inhibition activity of the compounds illustrated in the examples above is determined by the following methods. The COX-2 inhibition activity of the other COX-2 inhibitors of the present invention may also be determined by the following methods.

Step 1: Preparation of Recombinant COX Baculoviruses

Recombinant COX-1 and COX-2 are prepared as described by Gierse, et al., J. Biochem. 305:479-84 (1995). A 2.0 kb fragment containing the coding region of either human or murine COX-1 or human or murine COX-2 is cloned into a BamH1 site of the baculovirus transfer vector pVL1393 (Invitrogen) to generate the baculovirus transfer vectors for COX-1 and COX-2 in a manner similar to the method of O'Reilly et al., Baculovirus Expression Vectors: A Laboratory Manual (1992). Recombinant baculoviruses are isolated by transfecting 4 micrograms of baculovirus transfer vector DNA into SF9 insect cells (2×108) along with 200 ng of linearized baculovirus plasmid DNA by the calcium phosphate method. See Summers, et al., A Manual of Methods for Baculovirus Vectors and Insect Cell Culture Procedures, Texas Agric. Exp. Station Bull. 1555 (1987). Recombinant viruses are purified by three rounds of plaque purification and high titer (107-108 pfu/mL) stocks of virus are prepared. For large scale production, SF9 insect cells are infected in 10 liter fermentors (0.5×106/mL) with the recombinant baculovirus stock such that the multiplicity of infection is 0.1. After 72 hours the cells are centrifuged and the cell pellet is homogenized in Tris/Sucrose (50 mM: 25%, pH 8.0) containing 1% 3-[(3-cholamidopropyl)-dimethylammonio]-1-propanesulfonate (CHAPS). The homogenate is centrifuged at 10,000×G for 30 minutes, and the resultant supernatant is stored at −80° C. before being assayed for COX activity.

Step 2: Assay for COX-1 and COX-2 Activity

COX activity is assayed as PGE2 formed/microgram protein/time using an ELISA to detect the prostaglandin released. CHAPS-solubilized insect cell membranes containing the appropriate COX enzyme are incubated in a potassium phosphate buffer (50 mM, pH 8.0) containing epinephrine, phenol, and heme with the addition of arachidonic acid (10 micromolar). Compounds are pre-incubated with the enzyme for 10-20 minutes prior to the addition of arachidonic acid. Any reaction between the arachidonic acid and the enzyme is stopped after ten minutes at 37° C./room temperature by transferring 40 microliters of reaction mix into 160 microliters ELISA buffer and 25 micromolar indomethacin. The PGE2 formed is measured by standard ELISA technology (Cayman Chemical).

Step 3: Fast Assay for COX-1 and COX-2 Activity

COX activity is assayed as PGE2 formed/microgram protein/time using an ELISA to detect the prostaglandin released. CHAPS-solubilized insect cell membranes containing the appropriate COX enzyme are incubated in a potassium phosphate buffer (0.05 M Potassium phosphate, pH 7.5, 2 micromolar phenol, 1 micromolar heme, 300 micromolar epinephrine) with the addition of 20 microliters of 100 micromolar arachidonic acid (10 micromolar). Compounds are pre-incubated with the enzyme for 10 minutes at 25° C. prior to the addition of arachidonic acid. Any reaction between the arachidonic acid and the enzyme is stopped after two minutes at 37° C./room temperature by transferring 40 microliters of reaction mix into 160 microliters ELISA buffer and 25 micromolar indomethacin. The PGE2 formed is measured by standard ELISA technology (Cayman Chemical).

### Example 2

### LTB₄ Receptor Antagonist Activity

The LTB₄ activity of the compounds of the invention may be determined by comparing the ability of the compounds of the invention to compete with radiolabelled LTB₄ for specific LTB₄ receptor sites on guinea pig spleen membranes. Guinea pig spleen membranes are prepared as described by Chang et al. (J. Pharmacology and Experimental Therapeutics 232: 80, 1985). The ³H-LTB₄ binding assay is performed in 150 mg/liter containing 50 mM Tris pH 7.3, 10 mM MgCl₂, 9% Methanol, 0.7 nM ³H-LTB₄ (NEN, approximately 200 Ci/mmol) and 0.33 mg/ml guinea pig spleen membranes. Unlabeled LTB₄ is added at a concentration 5 micromolar to determine non-specific binding. Experimental compounds are added at varying concentrations to evaluate their effects on ³H-LTB₄ binding. The reactions are incubated at 4 degrees C. for 30 minutes. Membrane bound ³H-LTB₄ is collected by filtration through glass fiber filters and the amount bound is determined by scintillation counting. The IC₅₀ value for an experimental compound is the concentration at which 50% of specific ³H-LTB₄ binding is inhibited.

A combination therapy of a COX-2 inhibitor and an LTB₄ receptor antagonist can be evaluated as described in the following tests:

### Example 3

### Induction and Assessment of Collagen Induced Arthritis in Mice

A. Induction of Collagen-Induced Arthritis.

Arthritis is induced in 8-12 week old male DBA/1 mice by injection of 50 micrograms of chick type II collagen (CII) in complete Freunds adjuvant (Sigma) on day 0 at the base of the tail as described in J. Stuart, Annual Rev. Immunol., 2, 199 (1984). Compounds are prepared as a suspension in 0.5% methylcelluose (Sigma, St. Louis, Mo.), 0.025% Tween 20 (Sigma). The COX-2 inhibitors (Example 1 and 2) and LTB₄ receptor antagonist (Example 3) are administered alone or a COX-2 inhibitor and an LTB₄ receptor antagonist in combination. The compounds are administered in non-arthritic animals by gavage in a volume of 0.1 mL beginning on day 20 post collagen injection and continuing daily until final evaluation on day 55. Animals are boosted on day 21 with 50 micrograms of collagen (CII) in incomplete Freunds adjuvant. The animals are subsequently evaluated several times each week for incidence and severity of arthritis until day 56. Any animal with paw redness or swelling is counted as arthritic. Scoring of severity is carried out using a score of 0-3 for each paw (maximal score of 12/mouse) as described in P. Wooley, et al., Trans. Proc., 15, 180 (1983). The animals are measured for incidence of arthritis and severity in the animals where arthritis is observed. The incidence of arthritis is determined at a gross level by observing the swelling or redness in the paw or digits. Severity is measured with the following guidelines. Briefly, animals displaying four normal paws, i.e., no redness or swelling are scored 0. Any redness or swelling of digits or the paw is scored as 1. Gross swelling of the whole paw or deformity is scored as 2. Ankylosis of joints is scored as 3.

B. Histological Examination of Paws

In order to verify the gross determination of a non-arthritic animal, a histological examination is performed. Paws from animals sacrificed at the end of the experiment are removed, fixed and decalcified as previously described [R. Jonsson, J. Immunol. Methods, 88, 109 (1986)]. Samples are paraffin embedded, sectioned, and stained with hematoxylin and eosin by standard methods. Stained sections are examined for cellular infiltrates, synovial hyperplasia, and bone and cartilage erosion.

C. Animal Dose Ranges

The animals are dosed at one of the following dosing ranges:
1. 4-[5-(4-Chlorophenyl)-3-(trifluoromethyl)-1H-pyrazol-1-yl]benzenesulfonamide about 3 mpk/day; 7-[3-[2-(Cyclopropylmethyl)-3-methoxy-4-[(methylamino)carbonyl]phenoxy]propoxy]-3,4-dihydro-8-propyl-2H-1-benzopyran-2-propanoic acid about 3 mpk/day;
2. 4-[5-(3-Fluoro-4-methoxyphenyl)-3-(difluoromethyl)-1H-pyrazol-1-yl]benzenesulfonamide about 30 mpk/day; 7-[3-[2-(Cyclopropylmethyl)-3-methoxy-4-[(methylamino)carbonyl]phenoxy]-propoxy]-3,4-dihydro-8-propyl-2H-1-benzopyran-2-propanoic acid about 10 mpk/day;
3. 4-[5-(3-Fluoro-4-methoxyphenyl)-3-(difluoromethyl)-1H-pyrazol-1-yl]benzenesulfonamide about 10 mpk/day; 7-[3-[2-(Cyclopropylmethyl)-3-methoxy-4-[(methylamino)carbonyl]phenoxy]-propoxy]-3,4-dihydro-8-propyl-2H-1-benzopyran-2-propanoic acid about 10 mpk/day;
4. 4-[5-(4-Chlorophenyl)-3-(trifluoromethyl)-1H-pyrazol-1-yl]benzenesulfonamide Monday, Wednesday, and Friday about 10 mpk/day; 7-[3-[2-(Cyclopropylmethyl)-3-methoxy-4-[(methylamino)carbonyl]phenoxy]-propoxy]-3,4-dihydro-8-propyl-2H-1-benzopyran-2-propanoic acid about 10 mpk/day.
5. 7-[3-[2-(Cyclopropylmethyl)-3-methoxy-4-[(methylamino)carbonyl]-phenoxy]propoxy]-3,4-dihydro-8-propyl-2H-1-benzopyran-2-propanoic acid were prepared as described in U.S. Pat. No. 5,310,951, hereby incorporated by reference.
6. 4-[5-(4-Chlorophenyl)-3-(trifluoromethyl)-1H-pyrazol-1-yl]benzene-sulfonamide were prepared as in U.S. Pat. No. 5,466,823, hereby incorporated by reference.
7. 4-[5-(3-Fluoro-4-methoxyphenyl)-3-(difluoromethyl)-1H-pyrazol-1-yl]benzenesulfonamide were prepared as in U.S. Pat. No. 5,466,823, hereby incorporated by reference.

D. Collagen-Induced Arthritis Experiment:

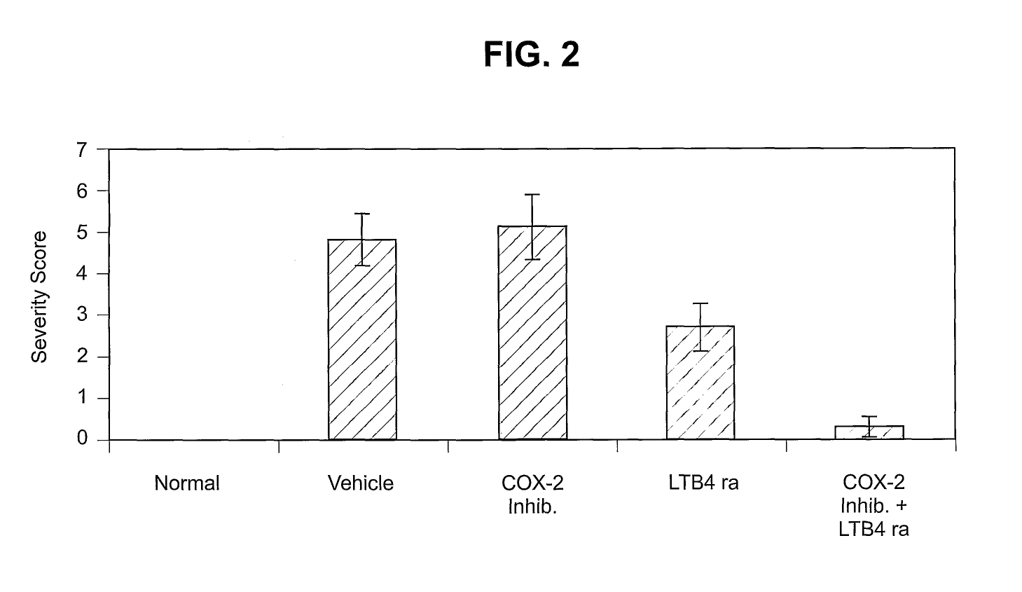

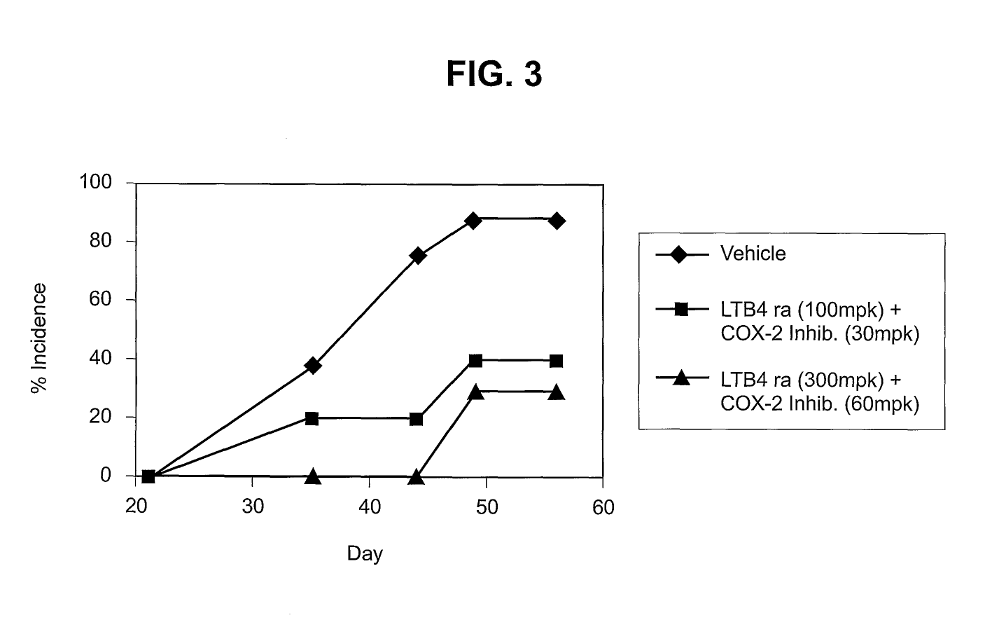

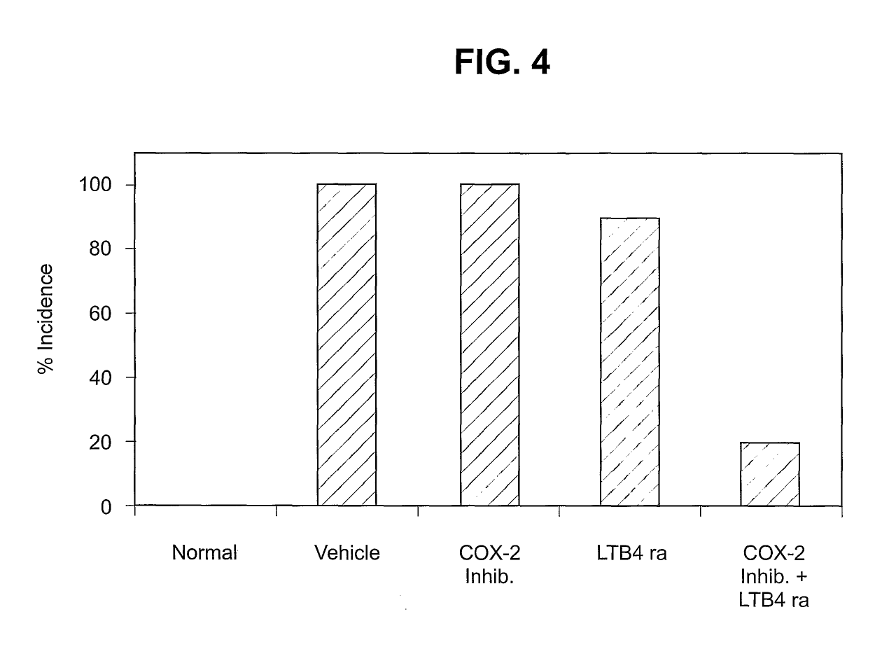

Eight- to twelve-week old male DBA/1 mice were injected at the base of the tail with 50 micrograms of chick type II collagen in CFA. 21 days later, animals were boosted with 50 micrograms chick type II collagen in IFA. Animals were also treated beginning on day 21 with Vehicle (0.5% Methylcellulose+0.025% Tween 80) or a COX-2 inhibitor (compound C-1, 15 mpk, bid), or an LTB₄ receptor antagonist (compound L-2, 150 mpk, bid), or the COX-2 inhibitor combined with the LTB₄ receptor antagonist. On day 56 animals were evaluated for arthritis. Any animals displaying inflammation in the paw were included as positive for incidence. In addition, paw swelling was graded on a standard scale of 0-3/paw (total score of 12/animal). Table 4 (corresponding to FIG. 2) shows the results of that experiment. A comparison of 2 LTB₄ receptor antagonist (compound L-2 and compound L-1) when combined with compound C-1 was made in the collagen induced arthritis model. Table 5 (corresponding to FIG. 3) shows the results of that study. Table 6 (corresponding to FIG. 4) shows the results of the individual inhibitors in that experiment.

TABLE 4

| Group | % Incidence | Severity | SEM | Weight | SEM |
|---|---|---|---|---|---|
| Normal | 0 | 0 | 0 | 27.94 | 0.728 |
| Vehicle | 100 | 4.8 | 0.611 | 24.06 | 0.417 |
| COX-2 Inhib. | 100 | 5.1 | 0.795 | 24.66 | 0.433 |
| LTB₄ ra | 90 | 2.7 | 0.559 | 26.77 | 0.574 |
| COX-2 Inhib. + LTB₄ ra | 20 | 0.333 | 0.238 | 26.33 | 0.484 |

SEM = Standard Error of the Mean

TABLE 5

% Incidence

| | | | LTB₄ ra | |
|---|---|---|---|---|
| | | LTB₄ ra | (300 mpk) + | |
| | | (100 mpk) + | COX-2 | |
| | | COX-2 | Inhib. | |
| Group | Vehicle | Inhib. (30 mpk) | (60 mpk) | No Rx |
| 21 | 0 | 0 | 0 | 0 |
| 35 | 38 | 20 | 0 | 90 |
| 44 | 75 | 20 | 0 | 100 |
| 49 | 88 | 40 | 29 | 100 |
| 56 | 88 | 40 | 29 | 100 |

TABLE 6

| Group | % Incidence | Severity | SEM | Weight | SEM |
|---|---|---|---|---|---|
| Normal | 0 | 0 | 0 | 27.94 | 0.728 |
| Vehicle | 100 | 4.8 | 0.611 | 24.06 | 0.417 |
| COX-2 Inhib. | 100 | 5.1 | 0.795 | 24.66 | 0.433 |
| LTB₄ ra | 90 | 2.7 | 0.559 | 26.77 | 0.574 |
| COX-2 Inhib. + LTB₄ ra | 20 | 0.333 | 0.238 | 26.33 | 0.484 |

SEM = Standard Error of the Mean

### Example 4

A formulation is prepared comprised of 700 mg of a COX-2 inhibitor and 700 mg of an LTB₄ receptor antagonist.

### Example 5

A formulation is prepared comprised of 350 mg of 4-[5-(3-fluoro-4-methoxyphenyl)-3-(difluoromethyl)-1H-pyrazol-1-yl]benzenesulfonamide and 350 mg of 7-[3-[2-(cyclopropylmethyl)-3-methoxy-4-[(methylamino)-carbonyl]phenoxy]propoxy]-3,4-dihydro-8-propyl-2H-1-benzopyran-2-propanoic acid.

### Example 6

### Solution ‘A’ Formulation of Compound L-1

A solution formulation is prepared to deliver Compound L-1 compound orally. A 25 mg/mL formulation is made by first adding L-1 in deionized water and then adjusting the pH to 7.0 with the addition of NaOH. Two percent (W/W) of polyvinylpyrrolidone with an average molecular weight of 10,000 is added into the solution as the polymeric precipitation inhibitor to decrease the likelihood of precipitation at lower pH environment in the GI tract upon oral administration. The above solution is used to prepare a C-1 suspension in order to co-administer both drugs: a formulation of 200 mg/mL C-1 suspension in 25 mg/mL L-1 solution is prepared by first making a 25 mg/mL L-1 solution following the above procedure. C-1 bulk drug is added and homogenized to ensure uniform particle size.

### Example 7

### Solution ‘B’ Formulation of C-1

A formulation of 200 mg/mL C-1 suspension in 25 mg/mL 2-[(3S,4R)-3,4-dihydro-4-hydroxy-3-(phenylmethyl)-2H-1-benzopyran-7-yl]-4-(trifluoromethyl)-benzoic acid solution is prepared by first adding the L-1 in deionized water and then adjusted the pH to 7.0 with the addition of NaOH. Two percent (w/w) of hydroxymethylcellulose (viscosity of 2% aqueous solution at 20° C., 40-60 centipoises) is dissolved into the solution along with 5% (w/w) Tween 80. Compound C-1 bulk drug is added and homogenized to ensure uniform particle size.

### Example 8

### Solution ‘C’ Formulation of Compound L-1

A formulation of 200 mg/mL compound C-1 suspension in 25 mg/mL L-1 solution is prepared by first adding L-1 in deionized water and then adjusted the pH to 7.0 with the addition of NaOH. Twenty percent (W/W) of PEG 400 is added followed by two percent (W/W) of hydroxymethylcellulose (viscosity of 2% aqueous solution at 20° C., 40-60 centipoises). Compound C-1 bulk drug is added and homogenized to ensure uniform particle size.

### Example 9

### Single-Layer Tablet with Two Drugs Granulated Together

| | Compound L-1/Compound C-1 dose | | | | | |
|---|---|---|---|---|---|---|
| | 200/200 mg | | | 200/100 mg | | |
| | | mg per | g per 1000 | | mg per | g per 1000 |
| Ingredients | % w/w | Tablet | tablets | % w/w | Tablet | tablets |
| Compound^{a}L-1 | 28.57 | 200.00 | 200.00 | 33.33 | 200.00 | 200.00 |
| Compound C-1 | 28.57 | 200.00 | 200.00 | 16.67 | 100.00 | 100.00 |
| ^{b,c}Lactose | 23.36 | 163.50 | 163.50 | 30.50 | 183.00 | 183.00 |
| Sodium Lauryl Sulfate | 3.00 | 21.00 | 21.00 | 3.00 | 18.00 | 18.00 |
| (SLS) | | | | | | |
| ^{c}Polyvinyl-pyrrolidone (PVP) | 2.50 | 17.50 | 17.50 | 2.50 | 15.00 | 15.00 |
| ^{d}Croscarmellose Sodium | 3.00 | 21.00 | 21.00 | 3.00 | 18.00 | 18.00 |
| ^{c}Microcrystalline Cellulose | 10.00 | 70.00 | 70.00 | 10.00 | 60.00 | 60.00 |
| Magnesium Stearate | 1.00 | 7.00 | 7.00 | 1.00 | 6.00 | 6.00 |
| To make | 100.00 | 700.00 | 700.00 | 100.00 | 600.00 | 600.00 |

^{a}Compound C-1 may be used as free acid or a salt; if salt is used, total weight of the tablet is increased or the amount of filler (e.g. lactose) is adjusted to accommodate the weight of the counter ion.
^{b}Lactose may be replaced by other water-soluble excipients like mannitol.
^{c}Polyvinylpyrrolidone may be replaced by other suitable binders like hydroxypropyl cellulose or hydroxypropyl methylcellulose, and amounts of microcrystalline cellulose and lactose may be modified to modulate the drug release profile.
^{d}Croscarmellose sodium may be replaced by other disintegrants like sodium starch glycolate or cross-linked polyvinylpyrrolidone, or eliminated for tablets requiring controlled release of the two drugs.

Compound L-1, compound C-1, lactose, SLS, PVP, croscarmellose sodium (portion of total or all), and microcrystalline cellulose are blended together and granulated using a dry granulation or a wet granulation process. For wet granulation, part or all of the SLS and/or PVP are not blended initially with the drug, and are added during the granulation step as a solution. The granules are delumped if necessary and are dried (for wet granulation). The dried granules are milled, blended with part or all of croscarmellose sodium, followed by final blending with magnesium stearate. The final blend is then compressed into tablets of appropriate size to get the desired dose.

Two tablets containing 200 mg compound L-1 and 100 mg compound C-1 are administered for the dose of 400 mg compound C-1 and 200 compound C-1.

### Example 10

### Bilayer Tablet (Compound L-1 Controlled Release/Compound C-1 Immediate Release)

| Compound L-1 (200 mg) Layer | | | | Compound C-1 (200 mg) Layer | | | |
|---|---|---|---|---|---|---|---|
| | | mg per | g per 1000 | | | mg per | g per 1000 |
| Ingredients | % w/w | Tablet | tablets | Ingredients | % w/w | Tablet | tablets |
| Compound ^{a}L-1 | 33.33 | 200.00 | 200.00 | Compound C-1 | 40.00 | 200.00 | 200.00 |
| ^{d}Poly- | 30.66 | 183.94 | 183.94 | ^{b,c}Lactose | 40.75 | 203.75 | 203.75 |
| ethylene | | | | | | | |
| oxide | | | | | | | |
| ^{b}Lactose | 17.00 | 102.00 | 102.00 | Sodium Lauryl | 3.00 | 15.00 | 15.00 |
| | | | | Sulfate | | | |
| Sodium chloride | 17.00 | 102.00 | 102.00 | ^{c}Polyvinyl- | 2.50 | 12.50 | 12.50 |
| | | | | pyrrolidone (PVP) | | | |
| Butylated | 0.01 | 0.06 | 0.06 | ^{e}Croscar-mellose | 3.00 | 15.00 | 15.00 |
| hydroxy-toluene | | | | Sodium | | | |
| Silicon dioxide | 0.50 | 3.00 | 3.00 | ^{c}Micro-crystalline | 10.00 | 50.00 | 50.00 |
| | | | | Cellulose | | | |
| Magnesium | 1.50 | 9.00 | 9.00 | Magnesium | 0.75 | 3.75 | 3.75 |
| Stearate | | | | Stearate | | | |
| To make | 100.00 | 600.00 | 600.00 | To make | 100.00 | 500.00 | 500.00 |

^{a}Compound L-1 may be used as free acid or a salt; if salt is used, total weight of the tablet will increase to accommodate the weight of the counter ion.
^{b}Lactose may be replaced by other water-soluble excipients like mannitol, polyethylene glycol, or sodium chloride.
^{c}Polyvinylpyrrolidone may be replaced by other suitable binders like hydroxypropyl cellulose or hydroxypropyl methylcellulose, and amounts of microcrystalline cellulose and lactose may be modified to modulate the drug release profile.
^{d}Polyethylene oxide may be a mixture of more than one molecular weight grade ranging from molecular weight of 200,000 to 5 million.
^{e}Croscarmellose sodium may be replaced by other disintegrants like sodium starch glycolate or cross-linked polyvinylpyrrolidone.

The components of the two layers are granulated separately.

For the compound L-1 layer, all ingredients except silicon dioxide and magnesium stearate are granulated using dry or wet granulation process. The granulation is dried (if wet granulated), milled, and blended with silicon dioxide, followed by final blending with magnesium stearate.

For the compound C-1 layer, all ingredients except part of croscarmellose sodium and magnesium stearate, are blended together and granulated using a dry granulation or a wet granulation process. For wet granulation, part or all of the SLS and/or PVP are not blended initially with the drug, and are added during the granulation step as a solution. The granules are delumped if necessary and are dried (for wet granulation). The dried granules are milled, blended with part or all of croscarmellose sodium, followed by final blending with magnesium stearate.

The two final blends are then compressed into bilayer tablets of appropriate size to get the desired dose.

Bilayer tablets with a total weight of 1100 mg (containing 600 mg of compound L-1 granulation and 500 mg of compound C-1 granulation) are made for the dose of 200 mg compound L-1 and 200 mg compound C-1.

Bilayer tablets with a total weight of 550 mg (containing 300 mg of Compound L-1 granulation and 250 mg of Compound C-1 granulation) are made for the dose of 100 mg Compound L-1 and 100 mg Compound C-1.

### Example 11

### Bilayer Tablet (Compound L-1 Controlled Release/Compound C-1 Immediate Release)

| Compound L-1 (200 mg) Layer | | | | Compound C-1 (100 mg) Layer | | | |
|---|---|---|---|---|---|---|---|
| | | mg per | g per 1000 | | | mg per | g per 1000 |
| Ingredients | % w/w | Tablet | tablets | Ingredients | % w/w | Tablet | tablets |
| Compound^{a} L-1 | 33.33 | 200.00 | 200.00 | Compound C-1 | 25.00 | 100.00 | 100.00 |
| ^{d}Poly-ethylene | 30.66 | 183.94 | 183.94 | ^{b,c}Lactose | 53.25 | 213.00 | 213.00 |
| oxide | | | | | | | |
| Lactose^{b} | 17.00 | 102.00 | 102.00 | Sodium Lauryl | 3.00 | 12.00 | 12.00 |
| | | | | Sulfate | | | |
| Sodium chloride | 17.00 | 102.00 | 102.00 | ^{c}Polyvinyl- | 2.50 | 10.00 | 10.00 |
| | | | | pyrrolidone (PVP) | | | |
| Butylated hydroxy- | 0.01 | 0.06 | 0.06 | ^{e}Croscar-mellose | 3.00 | 12.00 | 12.00 |
| toluene | | | | Sodium | | | |
| Silicon dioxide | 0.50 | 3.00 | 3.00 | ^{c}Micro-crystalline | 12.50 | 50.00 | 50.00 |
| | | | | Cellulose | | | |
| Magnesium | 1.50 | 9.00 | 9.00 | Magnesium | 0.75 | 3.00 | 3.00 |
| Stearate | | | | Stearate | | | |
| To make | 100.00 | 600.00 | 600.00 | To make | 100.00 | 400.00 | 400.00 |

^{a}Compound L-1 may be used as free acid or a salt; if salt is used, total weight of the tablet will increase to accommodate the weight of the counter ion.
^{b}Lactose may be replaced by other water-soluble excipients like mannitol, polyethylene glycol, or sodium chloride.
^{c}Polyvinylpyrrolidone may be replaced by other suitable binders like hydroxypropyl cellulose or hydroxypropyl methylcellulose, and amounts of microcrystalline cellulose and lactose may be modified to modulate the drug release profile.
^{d}Polyethylene oxide may be a mixture of more than one molecular weight grade ranging from molecular weight of 200,000 to 5 million.
^{e}Croscarmellose sodium may be replaced by other disintegrants like sodium starch glycolate or cross-linked polyvinylpyrrolidone.

The components of the two layers are granulated separately.

For the LTB4 compound L-1 layer, all ingredients except silicon dioxide and magnesium stearate are granulated using dry or wet granulation process. The granulation is dried (if wet granulated), milled, and blended with silicon dioxide, followed by final blending with magnesium stearate.

For COX-2 inhibitor C-1 layer, all ingredients except part of croscarmellose sodium and magnesium stearate, are blended together and granulated using a dry granulation or a wet granulation process. For wet granulation, part or all of the SLS and/or PVP are not blended initially with the drug, and are added during the granulation step as a solution. The granules are delumped if necessary and are dried (for wet granulation). The dried granules are milled, blended with part or all of croscarmellose sodium, followed by final blending with magnesium stearate.

The two final blends are then compressed into bilayer tablets of appropriate size to get the desired dose.

Bilayer tablets with a total weight of 1000 mg (containing 600 mg of compound L-1 granulation and 400 mg of Compound C-1 granulation) are made for the dose of 200 mg Compound L-1 and 100 mg Compound C-1.

Two tablets are administered for the dose of 400 mg compound L-1 and 200 mg compound C-1.

### Example 12

### Bilayer Tablet (Compound L-1 Controlled Release/Compound C-1 Immediate Release)

| Compound L-1 (200 mg) Layer | | | | Compound C-1 (200 mg) Layer | | | |
|---|---|---|---|---|---|---|---|
| | | mg per | g per 1000 | | | mg per | g per 1000 |
| Ingredients | % w/w | Tablet | tablets | Ingredients | % w/w | Tablet | tablets |
| Compound^{a}L-1 | 36.36 | 200.00 | 200.00 | Compound C-1 | 40.00 | 200.00 | 200.00 |
| ^{d}Hydroxy- | 45.00 | 247.50 | 247.50 | ^{b,c}Lactose | 40.75 | 203.75 | 203.75 |
| propyl methyl- | | | | | | | |
| cellulose | | | | | | | |
| ^{b}Lactose, | 17.14 | 94.25 | 94.25 | Sodium Lauryl | 3.00 | 15.00 | 15.00 |
| | | | | Sulfate | | | |
| Magnesium | 1.50 | 8.25 | 8.25 | ^{c}Polyvinyl- | 2.50 | 12.50 | 12.50 |
| Stearate | | | | pyrrolidone (PVP) | | | |
| — | — | — | — | ^{e}Croscar-mellose | 3.00 | 15.00 | 15.00 |
| | | | | Sodium | | | |
| — | — | — | — | ^{c}Micro-crystalline | 10.00 | 50.00 | 50.00 |
| | | | | Cellulose | | | |
| — | — | — | — | Magnesium | 0.75 | 3.75 | 3.75 |
| | | | | Stearate | | | |
| To make | 100.00 | 550.00 | 550.00 | To make | 100.00 | 500.00 | 500.00 |

^{a}Compound L-1 may be used as free acid or a salt; if salt is used, total weight of the tablet is increased or the amount of filler (e.g. lactose) is adjusted to accommodate the weight of the counter ion.
^{b}Lactose may be replaced by other water-soluble excipients like mannitol.
^{c}Polyvinylpyrrolidone may be replaced by other suitable binders like hydroxypropyl cellulose or hydroxypropyl methylcellulose, and amounts of microcrystalline cellulose and lactose may be modified to modulate the drug release profile.
^{d}Hydroxypropyl methylcellulose and lactose content may be modified in the L-1 layer to modulate the release profile.
^{e}Croscarmellose sodium may be replaced by other disintegrants like sodium starch glycolate or cross-linked polyvinylpyrrolidone.

The components of the two layers are granulated separately.

For the LTB4 L-1 compound layer, all ingredients except magnesium stearate are granulated using dry or wet granulation process. The granulation is dried (if wet granulated), milled, and blended with magnesium stearate to obtain final blend.

For C-1 layer, all ingredients except part of croscarmellose sodium and magnesium stearate, are blended together and granulated using a dry granulation or a wet granulation process. For wet granulation, part or all of the SLS and/or PVP are not blended initially with the drug, and are added during the granulation step as a solution. The granules are delumped if necessary and are dried (for wet granulation). The dried granules are milled, blended with part or all of croscarmellose sodium, followed by final blending with magnesium stearate.

The two final blends are then compressed into bilayer tablets of appropriate size to get the desired dose.

Bilayer tablets with a total weight of 1050 mg (containing 550 mg of Compound L-1 granulation and 500 mg of Compound C-1 granulation) are made for the dose of 200 mg Compound L-1 and 200 mg Compound C-1.

Bilayer tablets with a total weight of 525 mg (containing 275 mg of Compound L-1 granulation and 250 mg of Compound C-1 granulation) are made for the dose of 100 mg Compound L-1 and 100 mg Compound C-1.

### Example 13

### Bilayer Tablet (Compound L-1 Controlled Release/Compound C-1 Immediate Release)

| Compound L-1 (200 mg) Layer | | | | Compound C-1 (100 mg) Layer | | | |
|---|---|---|---|---|---|---|---|
| | | mg per | g per 1000 | | | mg per | g per |
| Ingredients | % w/w | Tablet | tablets | Ingredients | % w/w | Tablet | 1000 tablets |
| Compound^{a}L-1 | 36.36 | 200.00 | 200.00 | Compound C-1 | 25.00 | 100.00 | 100.00 |
| ^{d}Hydroxy-propyl | 45.00 | 247.50 | 247.50 | ^{b,c}Lactose | 53.25 | 213.00 | 213.00 |
| methyl-cellulose | | | | | | | |
| ^{b}Lactose | 17.14 | 94.25 | 94.25 | Sodium Lauryl | 3.00 | 12.00 | 12.00 |
| | | | | Sulfate | | | |
| Magnesium | 1.50 | 8.25 | 8.25 | ^{c}Polyvinyl- | 2.50 | 10.00 | 10.00 |
| Stearate | | | | pyrrolidone (PVP) | | | |
| — | — | — | — | ^{e}Croscar-mellose | 3.00 | 12.00 | 12.00 |
| | | | | Sodium | | | |
| — | — | — | — | ^{c}Micro-crystalline | 12.50 | 50.00 | 50.00 |
| | | | | Cellulose | | | |
| — | — | — | — | Magnesium | 0.75 | 3.00 | 3.00 |
| | | | | Stearate | | | |
| To make | 100.00 | 550.00 | 550.00 | To make | 100.00 | 400.00 | 400.00 |

^{a}Compound L-1 may be used as free acid or a salt; if salt is used, total weight of the tablet is increased or the amount of filler (e.g. lactose) is adjusted to accommodate the weight of the counter ion.
^{b}Lactose may be replaced by other water-soluble excipients like mannitol.
^{c}Polyvinylpyrrolidone may be replaced by other suitable binders like hydroxypropyl cellulose or hydroxypropyl methylcellulose, and amounts of microcrystalline cellulose and lactose may be modified to modulate the drug release profile.
^{d}Hydroxypropyl methylcellulose and lactose content may be modified in the compound L-1 layer to modulate the release profile.
^{e}Croscarmellose sodium may be replaced by other disintegrants like sodium starch glycolate or cross-linked polyvinylpyrrolidone.

The components of the two layers are granulated separately.

For the compound L-1 layer, all ingredients except magnesium stearate are granulated using dry or wet granulation process. The granulation is dried (if wet granulated), milled, and blended with magnesium stearate to obtain final blend.

For compound C-1 layer, all ingredients except part of croscarmellose sodium and magnesium stearate, are blended together and granulated using a dry granulation or a wet granulation process. For wet granulation, part or all of the SLS and/or PVP are not blended initially with the drug, and are added during the granulation step as a solution. The granules are delumped if necessary and are dried (for wet granulation). The dried granules are milled, blended with part or all of croscarmellose sodium, followed by final blending with magnesium stearate.

The two final blends are then compressed into bilayer tablets of appropriate size to get the desired dose.

Bilayer tablets with a total weight of 950 mg (containing 550 mg of compound L-1 granulation and 400 mg of compound C-1 granulation) are made for the dose of 200 mg compound L-1 and 100 mg compound C-1.

Two tablets are administered for the dose of 400 mg compound L-1 and 200 mg compound C-1.

### Example 14

### Multiparticulates (as Sachet)

| Compound L-1 (200 mg) beads (microspheres) | | | | Compound C-1 (200 mg) Granules | | | |
|---|---|---|---|---|---|---|---|
| | | mg per | g per 1000 | | | mg per | g per 1000 |
| Ingredients | % w/w | unit dose | unit doses | Ingredients | % w/w | unit dose | unit doses |
| Compound ^{a}L-1 | 40.00 | 200.00 | 200.00 | Compound C-1 | 62.50 | 200.00 | 200.00 |
| ^{b}Glyceryl | 57.00 | 285.00 | 285.00 | ^{c,d}Lactose | 19.00 | 60.80 | 60.80 |
| behenate | | | | | | | |
| ^{b}Polyoxy- | 3.00 | 15.00 | 15.00 | Sodium Lauryl | 3.00 | 9.60 | 9.60 |
| ethylene- | | | | Sulfate | | | |
| polyoxy- | | | | | | | |
| propylene | | | | | | | |
| copolymer | | | | | | | |
| | | | | ^{d}Polyvinyl- | 2.50 | 8.00 | 8.00 |
| | | | | pyrrolidone (PVP) | | | |
| — | — | — | — | ^{e}Croscar-mellose | 3.00 | 9.60 | 9.60 |
| | | | | Sodium | | | |
| — | — | — | — | ^{d}Micro-crystalline | 10.00 | 32.00 | 32.00 |
| | | | | Cellulose | | | |
| To make | 100.00 | 500.00 | 500.00 | To make | 100.0 | 320.00 | 320.00 |

^{a}Compound L-1 may be used as free acid or a salt; if salt is used, the total weight of the beads per dose will increase to accommodate the weight of the counter ion.
^{b}Polyoxyethylenepolyoxypropylene copolymer may be replaced by polyglycolyzed glycerides and/or glyceryl behenate content may be varied for modulating the drug release rates.
^{c}Lactose may be replaced by other water-soluble excipients like mannitol.
^{d}Polyvinylpyrrolidone may be replaced by other suitable binders like hydroxypropyl cellulose or hydroxypropyl methylcellulose, and amounts of microcrystalline cellulose and lactose may be modified to modulate the drug release profile.
^{e}Croscarmellose sodium may be replaced by other disintegrants like sodium starch glycolate or cross-linked polyvinylpyrrolidone.

For compound L-1 multiparticulates, all ingredients are blended together, followed by preparation of multiparticulates (or microspheres) using melt spray congealing process. These multiparticulates are then used “as is” or are coated using sustained release polymer (such as ethylcellulose, blend of cellulose acetate and cellulose acetate phthalate) or enteric coating polymer (such as hydroxypropyl methylcellulose phthalate, or methacrylic acid copolymer) for modulating the release profile as necessary.

For compound C-1 granulation, all ingredients are blended together and granulated using a dry granulation or a wet granulation process. For wet granulation, part or all of the SLS and/or PVP are not blended initially with the drug, and are added during the granulation step as a solution. The granules are delumped if necessary and are dried (for wet granulation). The dried granules are milled to a desired particle size.

Compound L-1 microspheres (500 mg plus weight of coating polymer) and compound C-1 granules (320 mg) are then mixed together, and 820 mg (plus additional weight to account for weight of polymer coating) of this mixture is prepared as a sachet for the dose of 200 mg compound L-1 and 200 mg compound C-1. An amount of 410 mg (plus additional weight to account for weight of polymer coating) of this mixture is used for the lower dose of 100 mg compound L-1 and 100 mg compound C-1.

As an alternative to a sachet, the mixture can be filled in capsules and administered as a capsule for lower doses where acceptable size capsule can accommodate the mixture.

### Example 15

### Multiparticulates (as a Sachet)

| Compound L-1 (200 mg) beads (microspheres) | | | | Compound C-1 (100 mg) Granules | | | |
|---|---|---|---|---|---|---|---|
| | | mg per | g per 1000 | | | mg per | g per 1000 |
| Ingredients | % w/w | unit dose | unit doses | Ingredients | % w/w | unit dose | unit doses |
| Compound^{a}L-1 | 40.00 | 200.00 | 200.00 | Compound C-1 | 40.00 | 100.00 | 100.00 |
| ^{b}Glyceryl | 57.00 | 285.00 | 285.00 | ^{c,d}Lactose | 40.50 | 101.25 | 101.25 |
| behenate | | | | | | | |
| ^{b}Polyoxy- | 3.00 | 15.00 | 15.00 | Sodium Lauryl | 3.00 | 7.50 | 7.50 |
| ethylene- | | | | Sulfate | | | |
| polyoxy- | | | | | | | |
| propylene | | | | | | | |
| copolymer | | | | | | | |
| | | | | ^{d}Polyvinyl- | 2.50 | 6.25 | 6.25 |
| | | | | pyrrolidone (PVP) | | | |
| — | — | — | — | ^{e}Croscar-mellose | 3.00 | 7.50 | 7.50 |
| | | | | Sodium | | | |
| — | — | — | — | ^{d}Micro-crystalline | 11.00 | 27.50 | 27.50 |
| | | | | Cellulose | | | |
| To make | 100.00 | 500.00 | 500.00 | To make | 100.00 | 250.00 | 250.00 |

^{a}Compound L-1 may be used as free acid or a salt; if salt is used, the total weight of the beads per dose will increase to accommodate the weight of counter ion.
^{b}Polyoxyethylene-polyoxypropylene copolymer may be replaced by polyglycolyzed glycerides and/or glyceryl behenate content may be varied for modulating the drug release rates.
^{c}Lactose may be replaced by other water-soluble excipients like mannitol.
^{d}Polyvinylpyrrolidone may be replaced by other suitable binders like hydroxypropyl cellulose or hydroxypropyl methylcellulose, and amounts of microcrystalline cellulose and lactose may be modified to modulate the drug release profile.
^{e}Croscarmellose sodium may be replaced by other disintegrants like sodium starch glycolate or cross-linked polyvinylpyrrolidone.

For compound L-1 multiparticulates, all ingredients are blended together, followed by preparation of multiparticulates (or microspheres) using melt spray congealing process. These multiparticulates are then used “as is” or are coated using sustained release polymer (such as ethylcellulose, blend of cellulose acetate and cellulose acetate phthalate) or enteric coating polymer (such as hydroxypropyl methylcellulose phthalate, or methacrylic acid copolymer) for modulating the release profile as necessary.

For compound C-1 granulation, all ingredients are blended together and granulated using a dry granulation or a wet granulation process. For wet granulation, part or all of the SLS and/or PVP are not blended initially with the drug, and are added during the granulation step as a solution. The granules are delumped if necessary and are dried (for wet granulation). The dried granules are milled to a desired particle size.

Compound L-1 microspheres (500 mg plus weight of coating polymer) and compound C-1 granules (250 mg) are then mixed together and 750 mg (plus additional weight to account for weight of polymer coating) of this mixture is dosed as a sachet for the dose of 200 mg compound L-1 and 100 mg of compound C-1. An amount of 1500 mg (plus additional weight to account for weight of polymer coating) of this mixture is used for the higher dose of 400 mg compound L-1 and 200 mg compound C-1.

As an alternative to a sachet, the mixture can be filled in capsules and administered as a capsule for lower doses where acceptable size capsule can accommodate the mixture.

### Example 16

### Capsules with Two Drugs Mixed Together in Granulation

| | Compound L-1/Compound C-1 | | | | | |
|---|---|---|---|---|---|---|
| | 200/200 mg | | | 200/100 mg | | |
| | | mg per | g per 1000 | | mg per | g per 1000 |
| Ingredients | % w/w | Capsule | capsules | % w/w | Capsule | capsules |
| Compound^{a} L-1 | 41.67 | 200.00 | 200.00 | 52.63 | 200.00 | 200.00 |
| Compound C-1 | 41.67 | 200.00 | 200.00 | 26.32 | 100.00 | 100.00 |
| ^{b,c}Lactose | 9.16 | 44.00 | 44.00 | 13.55 | 51.50 | 51.50 |
| ^{c}Sodium Lauryl Sulfate (SLS) | 3.00 | 14.40 | 14.40 | 3.00 | 11.40 | 11.40 |
| ^{c}Polyvinyl-pyrrolidone (PVP) | 2.50 | 12.00 | 12.00 | 2.50 | 9.50 | 9.50 |
| ^{d}Croscar-mellose Sodium | 1.00 | 4.80 | 4.80 | 1.00 | 3.80 | 3.80 |
| Magnesium Stearate | 1.00 | 4.80 | 4.80 | 1.00 | 3.80 | 3.80 |
| To make | 100.00 | 480.00 | 480.00 | 100.00 | 380.00 | 380.00 |

^{a}Compound L-1 may be used as free acid or a salt; if salt is used, total fill-weight of the capsule is increased or the amount of filler (e.g. lactose) is adjusted to accommodate the weight of the counter ion.
^{b}Lactose may be replaced by other water-soluble filler like mannitol, and its amount adjusted to achieve complete fill of the capsule.
^{c}Polyvinylpyrrolidone may be replaced by other suitable binders like hydroxypropyl cellulose, and amounts of lactose and SLS may be modified to modulate the drug release profile.
^{d}Croscarmellose sodium may be replaced by other disintegrants like sodium starch glycolate or cross-linked polyvinylpyrrolidone.

Compound L-1, compound C-1, lactose, SLS, PVP, and croscarmellose sodium (portion of total or all) are blended together and granulated using a dry granulation or a wet granulation process. For wet granulation, part or all of the SLS and/or PVP are not blended initially with the drug, and are added during the granulation step as a solution. The granules are delumped if necessary and are dried (for wet granulation). The dried granules are milled, blended with part or all of croscarmellose sodium, followed by final blending with magnesium stearate. The final blend is then filled into capsules of appropriate size and fill weight to get the desired dose.

Two capsules containing 200 mg compound L-1 and 100 mg compound C-1 are administered for the dose of 400 mg compound L-1 and 200 mg compound C-1.

### Example 17

### Single-Layer Tablet with Two Drugs Granulated Together

| | Compound L-1/Compound C-1 dose | | | | | |
|---|---|---|---|---|---|---|
| | 200/200 mg | | | 200/100 mg | | |
| | | mg per | g per 1000 | | mg per | g per 1000 |
| Ingredients | % w/w | Tablet | tablets | % w/w | Tablet | tablets |
| Compound^{a}L-1-EDA | 32.58 | 228.05 | 228.05 | 38.01 | 228.05 | 228.05 |
| Compound C-1 | 28.57 | 200.00 | 200.00 | 16.66 | 100.00 | 100.00 |
| ^{b,c}Mannitol | 29.35 | 205.45 | 205.45 | 35.83 | 214.95 | 214.95 |
| Sodium Lauryl Sulfate (SLS) | 3.00 | 21.00 | 21.00 | 3.00 | 18.00 | 18.00 |
| ^{c}Polyvinyl-pyrrolidone (PVP) | 2.50 | 17.50 | 17.50 | 2.50 | 15.00 | 15.00 |
| ^{d}Croscar-mellose Sodium | 3.00 | 21.00 | 21.00 | 3.00 | 18.00 | 18.00 |
| Magnesium Stearate | 1.00 | 7.00 | 7.00 | 1.00 | 6.00 | 6.00 |
| To make | 100.00 | 700.00 | 700.00 | 100.00 | 600.00 | 600.00 |

^{a}228.05 mg of Compound L-1-EDA (the ethylenediamine salt) is equivalent to 200 mg of Compound L-1 free acid.
^{b}Mannitol may be replaced by other water-soluble excipients. Other fillers may be added to facilitate compressibility.
^{c}Polyvinylpyrrolidone may be replaced by other suitable binder and amount of mannitol may be modified to modulate the drug release profile.
^{d}Croscarmellose sodium may be replaced by other disintegrants like sodium starch glycolate or cross-linked polyvinylpyrrolidone, or eliminated for tablets requiring controlled release of the two drugs.

Compound L-1-EDA, compound C-1, mannitol, SLS, PVP, and croscarmellose sodium (portion of total or all) are blended together and granulated using a dry granulation or a wet granulation process. For wet granulation, part or all of the SLS and/or PVP are not blended initially with the drug, and are added during the granulation step as a solution. The granules are delumped if necessary and are dried (for wet granulation). The dried granules are milled, blended with part or all of croscarmellose sodium, followed by final blending with magnesium stearate. The final blend is then compressed into tablets of appropriate size to get the desired dose. Two tablets containing 200 mg compound L-1 and 100 mg compound C-1 are administered for the dose of 400 mg compound L-1 and 200 mg compound C-1.

### Example 18

### Bilayer Tablet (Compound L-1 Controlled Release/Compound C-1 Immediate Release)

| Compound L-1 (200 mg) Layer | | | | Compound C-1 (200 mg) Layer | | | |
|---|---|---|---|---|---|---|---|
| | | mg per | g per 1000 | | | mg per | g per 1000 |
| Ingredients | % w/w | Tablet | tablets | Ingredients | % w/w | Tablet | tablets |
| Compound^{a}L-1- | 38.01 | 228.05 | 228.05 | Compound C-1 | 40.00 | 200.00 | 200.00 |
| EDA | | | | | | | |
| ^{d}Poly-ethylene | 30.66 | 183.94 | 183.94 | ^{b,c}Mannitol | 40.75 | 203.75 | 203.75 |
| oxide | | | | | | | |
| Mannitol^{b} | 14.33 | 86.00 | 86.00 | Sodium Lauryl | 3.00 | 15.00 | 15.00 |
| | | | | Sulfate | | | |
| Sodium chloride | 14.99 | 89.95 | 89.95 | ^{c}Polyvinyl- | 2.50 | 12.50 | 12.50 |
| | | | | pyrrolidone (PVP) | | | |
| Butylated | 0.01 | 0.060 | 0.060 | ^{e}Croscar-mellose | 3.00 | 15.00 | 15.00 |
| hydroxy-toluene | | | | Sodium | | | |
| Silicon dioxide | 0.50 | 3.00 | 3.00 | ^{c}Micro-crystalline | 10.00 | 50.00 | 50.00 |
| | | | | Cellulose | | | |
| Magnesium | 1.50 | 9.00 | 9.00 | Magnesium | 0.75 | 3.75 | 3.75 |
| Stearate | | | | Stearate | | | |
| To make | 100.00 | 600.00 | 600.00 | To make | 100.00 | 500.00 | 500.00 |

^{a}228.05 mg of compound L-1-EDA (the ethylenediamine salt) is equivalent to 200 mg of compound L-1 free acid.
^{b}Mannitol may be replaced by other water-soluble excipients like polyethylene glycol, or sodium chloride. Other fillers may be added to facilitate compressibility.
^{c}Polyvinylpyrrolidone may be replaced by other suitable binder, and amounts of microcrystalline cellulose and mannitol may be modified to modulate the drug release profile.
^{d}Polyethylene oxide may be a mixture of more than one molecular weight grade ranging from molecular weight of 200,000 to 5 million.
^{e}Croscarmellose sodium may be replaced by other disintegrants like sodium starch glycolate or cross-linked polyvinylpyrrolidone.

The components of the two layers are granulated separately.

For the L-1 layer, all ingredients except silicon dioxide and magnesium stearate are granulated using dry or wet granulation process. The granulation is dried (if wet granulated), milled, and blended with silicon dioxide, followed by final blending with magnesium stearate.

For the C-1 layer, all ingredients except part of croscarmellose sodium and magnesium stearate, are blended together and granulated using a dry granulation or a wet granulation process. For wet granulation, part or all of the SLS and/or PVP are not blended initially with the drug, and are added during the granulation step as a solution. The granules are delumped if necessary and are dried (for wet granulation). The dried granules are milled, blended with part or all of croscarmellose sodium, followed by final blending with magnesium stearate.

The two final blends are then compressed into bilayer tablets of appropriate size to get the desired dose.

Bilayer tablets with a total weight of 1100 mg (containing 600 mg of compound L-1 granulation and 500 mg of compound C-1 granulation) are made for the dose of 200 mg compound L-1 and 200 mg compound C-1.

Bilayer tablets with a total weight of 550 mg (containing 300 mg of compound L-1 granulation and 200 mg of compound C-1 granulation) are made for the dose of 100 mg compound L-1 and 100 mg compound C-1.

### Example 19

### Bilayer Tablet (Compound L-1 Controlled Release/Compound C-1 Immediate Release)

| Compound L-1 (200 mg) Layer | | | | Compound C-1 (100 mg) Layer | | | |
|---|---|---|---|---|---|---|---|
| | | mg per | g per 1000 | | | mg per | g per |
| Ingredients | % w/w | Tablet | tablets | Ingredients | % w/w | Tablet | 1000 tablets |
| Compound ^{a}L-1- | 38.01 | 228.05 | 228.05 | Compound C-1 | 25.00 | 100.00 | 100.00 |
| EDA | | | | | | | |
| ^{d}Poly-ethylene | 30.66 | 183.94 | 183.94 | ^{b,c}Mannitol | 53.25 | 213.00 | 213.00 |
| oxide | | | | | | | |
| Mannitol^{b} | 14.33 | 86.00 | 86.00 | Sodium Lauryl | 3.00 | 12.00 | 12.00 |
| | | | | Sulfate | | | |
| Sodium chloride | 14.99 | 89.95 | 89.95 | ^{c}Polyvinyl- | 2.50 | 10.00 | 10.00 |
| | | | | pyrrolidone (PVP) | | | |
| Butylated | 0.01 | 0.06 | 0.06 | ^{e}Croscar-mellose | 3.00 | 12.00 | 12.00 |
| hydroxy-toluene | | | | Sodium | | | |
| Silicon dioxide | 0.50 | 3.00 | 3.00 | ^{c}Micro-crystalline | 12.50 | 50.00 | 50.00 |
| | | | | Cellulose | | | |
| Magnesium | 1.50 | 9.00 | 9.00 | Magnesium | 0.75 | 3.00 | 3.00 |
| Stearate | | | | Stearate | | | |
| To make | 100.00 | 600.00 | 600.00 | To make | 100.00 | 400.00 | 400.00 |

^{a}228.05 mg of compound L-1-EDA (the ethylenediamine salt) is equivalent to 200 mg of compound L-1 free acid.
^{b}Mannitol may be replaced by other water-soluble excipients like polyethylene glycol, or sodium chloride. Other fillers may be added to facilitate compressibility.
^{c}Polyvinylpyrrolidone may be replaced by other suitable binder, and amounts of microcrystalline cellulose and mannitol may be modified to modulate the drug release profile.
^{d}Polyethylene oxide may be a mixture of more than one molecular weight grade ranging from molecular weight of 200,000 to 5 million.
^{e}Croscarmellose sodium may be replaced by other disintegrants like sodium starch glycolate or cross-linked polyvinylpyrrolidone.

The components of the two layers are granulated separately.

For the compound L-1 layer, all ingredients except silicon dioxide and magnesium stearate are granulated using dry or wet granulation process. The granulation is dried (if wet granulated), milled, and blended with silicon dioxide followed by final blending with magnesium stearate.

For the compound C-1 layer, all ingredients except part of croscarmellose sodium and magnesium stearate, are blended together and granulated using a dry granulation or a wet granulation process. For wet granulation, part or all of the SLS and/or PVP are not blended initially with the drug, and are added during the granulation step as a solution. The granules are delumped if necessary and are dried (for wet granulation). The dried granules are milled, blended with part or all of croscarmellose sodium, followed by final blending with magnesium stearate. The two final blends are then compressed into bilayer tablets of appropriate size to get the desired dose.

Bilayer tablets with a total weight of 1000 mg (containing 600 mg of compound L-1 granulation and 400 mg of compound C-1 granulation) are made for the dose of 200 mg compound L-1 and 100 mg compound C-1.

Two tablets are administered for the dose of 400 mg compound L-1 and 200 mg compound C-1.

### Example 20

### Multiparticulates (e.g., for a Sachet)

| Compound L-1 (200 mg) beads (microspheres) | | | | Compound C-1 (200 mg) Granules | | | |
|---|---|---|---|---|---|---|---|
| | | mg per | g per 1000 | | | mg per | g per 1000 |
| Ingredients | % w/w | unit dose | unit doses | Ingredients | % w/w | unit dose | unit doses |
| Compound ^{a}L-1- | 45.61 | 228.05 | 228.05 | Compound C-1 | 62.50 | 200.00 | 200.00 |
| EDA | | | | | | | |
| ^{b}Glyceryl | 51.39 | 256.95 | 256.95 | ^{c,d}Mannitol | 19.00 | 60.80 | 60.80 |
| behenate | | | | | | | |
| ^{b}Polyoxy- | 3.00 | 15.00 | 15.00 | Sodium Lauryl | 3.00 | 9.60 | 9.60 |
| ethylene-polyoxy- | | | | Sulfate | | | |
| propylene | | | | | | | |
| copolymer | | | | | | | |
| | | | | ^{d}Polyvinyl- | 2.50 | 8.00 | 8.00 |
| | | | | pyrrolidone (PVP) | | | |
| — | — | — | — | ^{e}Croscar-mellose | 3.00 | 9.60 | 9.60 |
| | | | | Sodium | | | |
| — | — | — | — | ^{d}Micro-crystalline | 10.00 | 32.00 | 32.00 |
| | | | | Cellulose | | | |
| To make | 100.00 | 500.00 | 500.00 | To make | 100.00 | 320.00 | 320.00 |

^{a}228.05 mg of compound L-1-EDA (the ethylenediamine salt) is equivalent to 200 mg of compound L-1 free acid.
^{b}Polyoxyethylene-polyoxypropylene copolymer may be replaced by polyglycolyzed glycerides and/or glyceryl behenate content may be varied for modulating the drug release rates.
^{c}Mannitol may be replaced by other water-soluble excipients.
^{d}Polyvinylpyrrolidone may be replaced by other suitable binder, and amounts of microcrystalline cellulose and mannitol may be modified to modulate the drug release profile.
^{e}Croscarmellose sodium may be replaced by other disintegrants like sodium starch glycolate or cross-linked polyvinylpyrrolidone.

For the compound L-1 multiparticulates, all ingredients are blended together, followed by preparation of multiparticulates (or microspheres) using melt spray congealing process. These multiparticulates are then used “as is” or are coated using sustained release polymer (such as ethylcellulose, blend of cellulose acetate and cellulose acetate phthalate) or enteric coating polymer (such as hydroxypropyl methylcellulose phtalate, or methacrylic acid copolymer) for modulating the release profile as necessary.

For compound C-1 granulation, all ingredients are blended together and granulated using a dry granulation or a wet granulation process. For wet granulation, part or all of the SLS and/or PVP are not blended initially with the drug, and are added during the granulation step as a solution. The granules are delumped if necessary and are dried (for wet granulation). The dried granules are milled to a desired particle size.

Compound L-1 microspheres (500 mg plus weight of coating polymer) and compound C-1 granules (320 mg) are then mixed together and 820 mg (plus additional weight to account for weight of polymer coating) of this mixture is dosed as sachet for the dose of 200 mg compound L-1 and 200 mg compound C-1. 410 mg (plus additional weight to account for weight of polymer coating) of this mixture is used for lower dose of 100 mg compound L-1 and 100 mg compound C-1.

As an alternative to sachet, the mixture can be filled in capsules and dosed as a capsule for lower doses where acceptable size capsule can accommodate the mixture.

### Example 21

### Multiparticulates (e.g., for a Sachet)

| Compound L-1 (200 mg) beads (microspheres) | | | | Compound C-1 (100 mg) Granules | | | |
|---|---|---|---|---|---|---|---|
| | | mg per | g per 1000 | | | mg per | g per 1000 |
| Ingredients | % w/w | unit dose | unit doses | Ingredients | % w/w | unit dose | unit doses |
| Compound | 45.61 | 228.05 | 228.05 | Compound C-1 | 40.00 | 100.00 | 100.00 |
| ^{a}L-1-EDA | | | | | | | |
| ^{b}Glyceryl | 51.39 | 256.95 | 256.95 | ^{c,d}Mannitol | 40.50 | 101.25 | 101.25 |
| behenate | | | | | | | |
| ^{b}Polyoxy- | 3.00 | 15.00 | 15.00 | Sodium Lauryl | 3.00 | 7.50 | 7.50 |
| ethylene-polyoxy- | | | | Sulfate | | | |
| propylene | | | | | | | |
| copolymer | | | | | | | |
| | | | | ^{d}Polyvinyl- | 2.50 | 6.25 | 6.25 |
| | | | | pyrrolidone (PVP) | | | |
| — | — | — | — | ^{e}Croscar-mellose | 3.00 | 7.50 | 7.50 |
| | | | | Sodium | | | |
| — | — | — | — | ^{d}Micro-crystalline | 11.00 | 27.50 | 27.50 |
| | | | | Cellulose | | | |
| To make | 100.00 | 500.00 | 500.00 | To make | 100.00 | 250.00 | 250.00 |

^{a}228.05 mg of compound L-1-EDA (the ethylenediamine salt) is equivalent to 200 mg of compound L-1 free acid.
^{b}Polyoxyethylene-polyoxypropylene copolymer may be replaced by polyglycolyzed glycerides and/or glyceryl behenate content may be varied for modulating the drug release rates.
^{c}Mannitol may be replaced by other water-soluble excipients.
^{d}Polyvinylpyrrolidone may be replaced by other suitable binder, and amounts of microcrystalline cellulose and mannitol may be modified to modulate the drug release profile.
^{e}Croscarmellose sodium may be replaced by other disintegrants like sodium starch glycolate or cross-linked polyvinylpyrrolidone.

For the compound L-1 multiparticulates, all ingredients are blended together, followed by preparation of multiparticulates (or microspheres) using melt spray congealing process. These multiparticulates are then used “as is” or are coated using sustained release polymer (such as ethylcellulose, blend of cellulose acetate and cellulose acetate phthalate) or enteric coating polymer (such as hydroxypropyl methylcellulose phtalate, or methacrylic acid copolymer) for modulating the release profile as necessary.

For compound C-1 granulation, all ingredients are blended together and granulated using a dry granulation or a wet granulation process. For wet granulation, part or all of the SLS and/or PVP are not blended initially with the drug, and are added during the granulation step as a solution. The granules are delumped if necessary and are dried (for wet granulation). The dried granules are milled to a desired particle size.

Compound L-1 microspheres (500 mg plus weight of coating polymer) and compound C-1 granules (250 mg) are then mixed together, and 750 mg (plus additional weight to account for weight of polymer coating) of this mixture is dosed as sachet for the dose of 200 mg compound L-1 and 100 mg compound C-1. 1500 mg (plus additional weight to account for weight of polymer coating) of this mixture is used for the higher dose of 400 mg compound L-1 and 200 mg compound C-1.

As an alternative to sachet, the mixture can be filled in capsules and dosed as a capsule for lower doses where acceptable size capsule can accommodate the mixture.

### Example 22

### Capsules with Two Drugs Mixed Together in Granulation

| | Compound L-1/Compound C-1 dose | | | | | |
|---|---|---|---|---|---|---|
| | 200/200 mg | | | 200/100 mg | | |
| | | mg per | g per 1000 | | mg per | g per 1000 |
| Ingredients | % w/w | Capsule | capsules | % w/w | Capsule | capsules |
| Compound ^{a}L-1-EDA | 45.61 | 228.05 | 228.05 | 60.01 | 228.05 | 228.05 |
| Compound C-1 | 40.00 | 200.00 | 200.00 | 26.32 | 100.00 | 100.00 |
| ^{b,c}Mannitol | 6.89 | 34.45 | 34.45 | 6.17 | 23.45 | 23.45 |
| Sodium Lauryl Sulfate (SLS) | 3.00 | 15.00 | 15.00 | 3.00 | 11.40 | 11.40 |
| ^{c}Polyvinyl-pyrrolidone (PVP) | 2.50 | 12.50 | 12.50 | 2.50 | 9.50 | 9.50 |
| ^{d}Croscar-mellose Sodium | 1.00 | 5.00 | 5.00 | 1.00 | 3.80 | 3.80 |
| Magnesium Stearate | 1.00 | 5.00 | 5.00 | 1.00 | 3.80 | 3.80 |
| To make | 100.00 | 500.00 | 500.00 | 100.00 | 380.00 | 380.00 |

^{a}228.05 mg of compound L-1-EDA (the ethylenediamine salt) is equivalent to 200 mg of Compound L-1 free acid.
^{b}Mannitol may be replaced by other water-soluble filler, and its amount adjusted to achieve complete fill of the capsule.
^{c}Polyvinylpyrrolidone may be replaced by other suitable binder, and amounts of mannitol and SLS may be modified to modulate the drug release profile.
^{d}Croscarmellose sodium may be replaced by other disintegrants like sodium starch glycolate or cross-linked polyvinylpyrrolidone.

Compound L-1-EDA, compound C-1, mannitol, SLS, PVP, and croscarmellose sodium (portion of total or all) are blended together and granulated using a dry granulation or a wet granulation process. For wet granulation, part or all of the SLS and/or PVP are not blended initially with the drug, and are added during the granulation step as a solution. The granules are delumped if necessary and are dried (for wet granulation). The dried granules are milled, blended with part or all of croscarmellose sodium, followed by final blending with magnesium stearate. The final blend is then filled into capsules of appropriate size and fill weight to get the desired dose.

Two capsules containing 200 mg compound L-1 and 100 mg compound C-1 are administered for the dose of 400 mg compound L-1 and 200 mg compound C-1.

The examples herein can be performed by substituting the generically or specifically described reactants and/or operating conditions of this invention for those used in the preceding examples.

The invention being thus described, it is apparent that the same can be varied in many ways. Such variations are not to be regarded as a departure from the spirit and scope of the present invention, and all such modifications and equivalents as would be obvious to one skilled in the art are intended to be included within the scope of the following claims.

### Example 23

### Bilayer Tablet

| Compound L-1 (200 mg) Layer | | | | Compound C-1 (200 mg) Layer | | | |
|---|---|---|---|---|---|---|---|
| | | mg per | g per 1000 | | | mg per | g per 1000 |
| Ingredients | % w/w | Tablet | tablets | Ingredients | % w/w | Tablet | tablets |
| Compound ^{a}L-1 | 40.00 | 200.00 | 200.00 | Compound C-1 | 40.00 | 200.00 | 200.00 |
| ^{b,c}Lactose | 42.00 | 210.00 | 210.00 | ^{b,c}Lactose | 40.75 | 203.75 | 203.75 |
| Sodium Lauryl | 1.00 | 5.00 | 5.00 | Sodium Lauryl | 3.00 | 15.00 | 15.00 |
| Sulfate (SLS) | | | | Sulfate (SLS) | | | |
| ^{c}Polyvinyl- | 2.50 | 12.50 | 12.50 | ^{c}Polyvinyl- | 2.50 | 12.50 | 12.50 |
| pyrrolidone (PVP) | | | | pyrrolidone (PVP) | | | |
| ^{d}Croscar-mellose | 3.00 | 15.00 | 15.00 | ^{d}Croscar-mellose | 3.00 | 15.00 | 15.00 |
| Sodium | | | | Sodium | | | |
| ^{c}Micro-crystalline | 10.00 | 50.00 | 50.00 | ^{c}Micro-crystalline | 10.00 | 50.00 | 50.00 |
| Cellulose | | | | Cellulose | | | |
| Magnesium | 1.50 | 7.50 | 7.50 | Magnesium | 0.75 | 3.75 | 3.75 |
| Stearate | | | | Stearate | | | |
| To make | 100.00 | 500.00 | 500.00 | To make | 100.00 | 500.00 | 500.00 |

^{a}Compound L-1 may be used as free acid or a salt; if salt is used, total weight of the tablet is increased or the amount of filler (e.g. lactose) is adjusted to accommodate the weight of the counter ion.
^{b}Lactose may be replaced by other water-soluble excipients like mannitol.
^{c}Polyvinylpyrrolidone may be replaced by other suitable binders like hydroxypropyl cellulose or hydroxypropyl methylcellulose, and amounts of binder, microcrystalline cellulose and lactose may be modified to modulate the drug release profile and the tablet compression characteristics.
^{d}Croscarmellose sodium may be replaced by other disintegrants like sodium starch glycolate or cross-linked polyvinylpyrrolidone, and the amounts may be modified to modulate the disintegration time.

The components of the two layers are granulated separately.

For compound L-1 layer, all ingredients except part of croscarmellose sodium and magnesium stearate, are blended together and granulated using a dry granulation or a wet granulation process. For wet granulation, part or all of the SLS and/or PVP are not blended initially with the drug, and are added during the granulation step as a solution. The granules are delumped if necessary and are dried (for wet granulation). The dried granules are milled, blended with part or all of croscarmellose sodium, followed by final blending with the remaining magnesium stearate.

For compound C-1 layer, all ingredients except part of croscarmellose sodium and magnesium stearate, are blended together and granulated using a dry granulation or a wet granulation process. For wet granulation, part or all of the SLS and/or PVP are not blended initially with the drug, and are added during the granulation step as a solution. The granules are delumped if necessary and are dried (for wet granulation). The dried granules are milled, blended with part or all of croscarmellose sodium, followed by final blending with the remaining magnesium stearate.

The two final blends are then compressed into bilayer tablets of appropriate size to get the desired dose.

Bilayer tablets with a total weight of 1000 mg (containing 500 mg of compound L-1 granulation and 500 mg of compound C-1 granulation) are made for the dose of 200 mg compound L-1 and 200 mg compound C-1.

Bilayer tablets with a total weight of 500 mg (containing 250 mg of compound L-1 granulation and 250 mg of compound C-1 granulation) are made for the dose of 100 mg compound L-1 and 100 mg compound C-1.

### Example 24

### Bilayer Tablet

| Compound L-1 (200 mg) Layer | | | | Compound C-1 (100 mg) Layer | | | |
|---|---|---|---|---|---|---|---|
| | | mg per | g per 1000 | | | mg per | g per 1000 |
| Ingredients | % w/w | Tablet | tablets | Ingredients | % w/w | Tablet | tablets |
| Compound ^{a}L-1 | 40.00 | 200.00 | 200.00 | Compound C-1 | 25.00 | 100.00 | 100.00 |
| ^{b,c}Lactose | 42.00 | 210.00 | 210.00 | ^{b,c}Lactose | 53.25 | 213.00 | 213.00 |
| Sodium Lauryl | 1.00 | 5.00 | 5.00 | Sodium Lauryl | 3.00 | 12.00 | 12.00 |
| Sulfate (SLS) | | | | Sulfate (SLS) | | | |
| ^{c}Polyvinyl- | 2.50 | 12.50 | 12.50 | ^{c}Polyvinyl- | 2.50 | 10.00 | 10.00 |
| pyrrolidone (PVP) | | | | pyrrolidone (PVP) | | | |
| ^{d}Croscar-mellose | 3.00 | 15.00 | 15.00 | ^{d}Croscar-mellose | 3.00 | 12.00 | 12.00 |
| Sodium | | | | Sodium | | | |
| ^{c}Micro-crystalline | 10.00 | 50.00 | 50.00 | ^{c}Micro-crystalline | 12.50 | 50.00 | 50.00 |
| Cellulose | | | | Cellulose | | | |
| Magnesium | 1.50 | 7.50 | 7.50 | Magnesium | 0.75 | 3.00 | 3.00 |
| Stearate | | | | Stearate | | | |
| To make | 100.00 | 500.00 | 500.00 | To make | 100.00 | 400.00 | 400.00 |

^{a}Compound L-1 may be used as free acid or a salt; if salt is used, total weight of the tablet is increased or the amount of filler (e.g. lactose) is adjusted to accommodate the weight of the counter ion.
^{b}Lactose may be replaced by other water-soluble excipients like mannitol.
^{c}Polyvinylpyrrolidone may be replaced by other suitable binders like hydroxypropyl cellulose or hydroxypropyl methylcellulose, and amounts of binder, microcrystalline cellulose and lactose may be modified to modulate the drug release profile and the tablet compression characteristics.
^{d}Croscarmellose sodium may be replaced by other disintegrants like sodium starch glycolate or cross-linked polyvinylpyrrolidone, and the amounts may be modified to modulate the disintegration time.

The components of the two layers are granulated separately.

For compound L-1 layer, all ingredients except part of croscarmellose sodium and magnesium stearate, are blended together and granulated using a dry granulation or a wet granulation process. For wet granulation, part or all of the SLS and/or PVP are not blended initially with the drug, and are added during the granulation step as a solution. The granules are delumped if necessary and are dried (for wet granulation). The dried granules are milled, blended with part or all of croscarmellose sodium, followed by final blending with the remaining magnesium stearate.

For compound C-1 layer, all ingredients except part of croscarmellose sodium and magnesium stearate, are blended together and granulated using a dry granulation or a wet granulation process. For wet granulation, part or all of the SLS and/or PVP are not blended initially with the drug, and are added during the granulation step as a solution. The granules are delumped if necessary and are dried (for wet granulation). The dried granules are milled, blended with part or all of croscarmellose sodium, followed by final blending with the remaining magnesium stearate.

The two final blends are then compressed into bilayer tablets of appropriate size to get the desired dose.

Bilayer tablets with a total weight of 900 mg (containing 500 mg of compound L-1 granulation and 400 mg of compound C-1 granulation) are made for the dose of 200 mg compound L-1 and 100 mg compound C-1.

Two tablets are administered for the dose of 400 mg compound L-1 and 200 mg compound C-1.

### Example 25

### Murine Air Pouch Model

Female Balb/c mice age 8-12 weeks are injected with approximately 5 milliliters of air subcutaneous using a 10 milliliter syringe with a 27-gauge needle and a 0.2 micron acrodisc filter. The animals are anesthetized with CO₂/O₂ mix, and then the air injection is made subcutaneously in the intrascapular region of the mouse. Animals' pouches are reinflated every 2-3 days with approximately 2-3 milliliters of air, same as above. Dosing of each compound, including Vehicle (0.5% Methylcellulose+0.025% Tween 80) or a COX-2 inhibitor (compound C-1, 15 mpk, BID), or an LTB₄ receptor antagonist (compound L-2, 150 mpk, BID), or the COX-2 inhibitor combined with the LTB₄ receptor antagonist (compound C-1, 30 mpk+compound L-2 150 mpk BID), starting on day 5 at 6 am. Compounds are given orally (PO) and dosed BID in a volume of 0.2 milliliters per/dose. On day 7 animals were again anesthetized with CO₂/O₂ and the air pouch is injected using a 27 guage needle and 3 milliliter syringe with stimulant, which is (1% zymosan Sigma Z-4250) made in saline solution (0.9% saline, Baxter #2f7122). Two hours post stimulation the mice are euthanized, and the pouches are lavaged with a set 1 milliliter volume of Dmem/F12 Gibco #21041-025. The cells are then placed in tubes on ice keeping each mouse lavage fluid separated. Cells are centrifuged at 1600 rpms for 10 minutes. Cells are then resuspended in 1 milliliter of the same media used to lavage cells. Total cells/mouse is calculated using a coulter counter. Results of this study are shown in Table 7, corresponding to FIG. 1.

TABLE 7

| | Total | Standard | |
|---|---|---|---|
| Group | Cells/Mouse | Deviation | SEM |
| Control | 51800000 | 179000 | 8000 |
| vehicle + Zymosan | 43000000 | 1410 | 812 |
| 30 mpk COX-2 | 40600000 | 16200 | 7230 |
| Inhibitor | | | |
| 300mpk LTB₄ RA | 38900000 | 22000 | 11000 |
| COX-2 Inhib + LTB₄ | 24300000 | 15000 | 7510 |
| RA | | | |

SEM = Standard Error of the Mean

What is claimed is:

1. A therapeutic composition comprising at least one COX-2 selective inhibitor or a prodrug thereof and at least one LTB₄ receptor antagonist wherein the LTB₄ receptor antagonist comprises one or more compounds selected from the group consisting of 2-[(3S,4R)-3,4-dihydro-4-hydroxy-3-(phenylmethyl)-2H-1-benzopyran-7-yl]-4-(trifluoromethyl)benzoic acid; a pharmaceutically-acceptable salt thereof; and mixtures thereof.

2. The therapeutic composition of claim 1, wherein the COX-2 selective inhibitor comprises celecoxib.

3. The therapeutic composition of claim 1 wherein the LTB₄ receptor antagonist comprises 2-[(3S,4R)-3,4-dihydro-4-hydroxy-3-(phenylmethyl)-2H-1-benzopyran-7-yl]-4-(trifluoromethyl)benzoic acid.

4. The therapeutic composition of claim 3 wherein the COX-2 selective inhibitor comprises celecoxib.

5. The therapeutic composition of claim 1 wherein the LTB₄ receptor antagonist comprises a pharmaceutically-acceptable salt of 2-[(3S,4R)-3,4-dihydro-4-hydroxy-3-(phenylmethyl)-2H-1-benzopyran-7-yl]-4-(trifluoromethyl)benzoic acid.

6. The therapeutic composition of claim 1 wherein the LTB₄ receptor antagonist comprises the mono(ethylene diamine) salt of 2-[(3S,4R)-3,4-dihydro-4-hydroxy-3-(phenylmethyl)-2H-1-benzopyran-7-yl]-4-(trifluoromethyl)benzoic acid.

7. The therapeutic composition of claim 6 wherein the COX-2 selective inhibitor comprises celecoxib.

8. The composition of claim 1 or 2, further comprising a pharmaceutically-acceptable excipient.

9. The composition of claim 1 or 2, wherein the composition is a solid dosage form.

10. The composition of claim 9, wherein the solid dosage form is an oral dosage form.

11. The composition of claim 10, wherein the oral dosage form is selected from a group consisting of a tablet, a capsule, a suppository, a pill, a gel cap, and a granular composition.

12. The composition of claim 11 wherein the oral dosage form is a capsule.

13. The composition of claim 12 wherein the capsule is a time release capsule dosage form.

14. The composition of claim 11 wherein the oral dosage form is a tablet dosage form.

15. The composition of 14 wherein the tablet dosage form is selected from a group consisting of a multiple layer tablet dosage form, a sustained release tablet dosage form, a core-mantle tablet dosage form, an osmotic tablet dosage form, and a side-by-side tablet dosage form.

16. The composition of claim 15 wherein the tablet dosage form comprises a multiple layer tablet dosage form.

17. The therapeutic composition of claim 15 wherein the tablet dosage form comprises a side-by-side tablet dosage form.

18. The composition of claim 15 wherein the tablet dosage form comprises a sustained release tablet dosage form.

19. The composition of claim 15 wherein the tablet dosage form comprises a core and mantle tablet dosage form.

20. The composition of claim 1 or 2, wherein the COX-2 selective inhibitor or a prodrug thereof and an LTB₄ receptor antagonist are present in an intimate mixture.

21. The composition of claim 1 or 2, wherein the composition is an aqueous form.

22. The composition of claim 21, wherein the aqueous form is a syrup.

23. The composition of claim 21, wherein the aqueous form is suitable for parenteral administration.

24. The composition of claim 1 or 2, wherein the composition is in an inhalable dosage form.

25. The composition of claim 1 or 2, wherein the composition is in a semi-solid dosage form.

26. The composition of claim 25, wherein the semi-solid form is suitable for topical application.

27. The composition of claim 1 or 2 wherein the composition is a suspension.

28. A method for the treatment, prevention, or inhibition of inflammation, an inflammation-related disorder, pain-related disorder, or pain in a subject in need of such prevention, treatment, or inhibition, the method comprising administering to the subject a composition comprising a COX-2 selective anti-inflammatory compound and an LTB₄ receptor antagonist compound, wherein the LTB₄ receptor antagonist compound comprises one or more compounds selected from the group consisting of 2-[(3S,4R)-3,4-dihydro-4-hydroxy-3-(phenylmethyl)-2H-1-benzopyran-7-yl]-4-(trifluoromethyl)benzoic acid; salts; and mixtures thereof.

29. The method of claim 28, wherein the COX-2 selective inhibitor comprises celecoxib.

30. The method of any one of claims 28 or 29, wherein the subject is an animal.

31. The method of claim 30, wherein the animal is a human.

32. The method of claim 31 for the treatment, prevention or inhibition of an inflammation-related disorder.

33. The method of claim 31 for the treatment, prevention or inhibition of inflammation.

34. The method of claim 31 for the treatment, prevention or inhibition of pain.

35. The method of claim 31 for the treatment, prevention or inhibition of a pain-related disorder.

36. The method of claim 31 wherein the inflammation-related disorder is arthritis.

37. The method of claim 36 wherein the arthritis is osteoarthritis.

38. The method of claim 37 wherein the arthritis is rheumatoid arthritis.

39. The method of claim 31, for the prevention or treatment of any one or more of the disorders selected from the group consisting of connective tissue and joint disorder, neoplasia disorder, cardiovascular disorder, otic disorder, ophthalmic disorder, respiratory disorder, gastrointestinal disorder, angiogenesis-related disorder, immunological disorders, allergic disorder, nutritional disorder, infectious disease and disorders, endocrine disorder, metabolic disorder, neurological and neurodegenerative disorder, psychiatric disorder, hepatic and biliary disorder, musculoskeletal disorder, genitourinary disorder, gynecologic and obstetric disorder, injury and trauma disorder, surgical disorder, dental and oral disorder, sexual dysfunction disorder, dermatologic disorder, hematological disorder, and poisoning disorder.

40. The method of claim 28 wherein the amount of LTB₄ receptor antagonist, and the amount of COX-2 selective inhibitor, are administered in a sequential manner.

41. The method of claim 28 wherein the amount of LTB₄ receptor antagonist and the amount of COX-2 selective inhibitor are administered in a substantially simultaneous manner.
